(19) United States
(12) Patent Application Publication
(10) Pub. No.: US 2026/0083776 A1
(43) Pub. Date: Mar. 26, 2026

(54) ACTIVATION AND PROLIFERATION OF CYTOTOXIC LYMPHOCYTES

(71) Applicant: B.G. NEGEV TECHNOLOGIES AND APPLICATIONS LTD.,AT BEN-GURION UNIVERSITY, Beer Sheva (IL)

(72) Inventors: Mark SCHVARTZMAN, Herzliya (IL); Angel PORGADOR, Lehavim (IL); Ashish PANDEY, New York, NY (US); Carlos Ureña MARTIN, Barcelona (ES); Guillaume LE SAUX, Villenave d'Ornon (FR)

(21) Appl. No.: 19/108,842

(22) PCT Filed: Sep. 5, 2023
(86) PCT No.: PCT/IL2023/050955

§ 371 Date: Mar. 5, 2025

Related U.S. Application Data
(60) Provisional application No. 63/403,881, filed on Sep. 6, 2022.
(60) Provisional application No. 63/452,452, filed on Mar. 16, 2023.
(60) Provisional application No. 63/528,384, filed on Jul. 23, 2023.

(51) Int. Cl. A61K 35/17; A61K 40/11; A61K 40/31; A61P 35/00; C12N 5/00; C12N 5/0783; C12N 9/22; C12N 15/11; C12N 15/87

(52) U.S. Cl. CPC A61K 35/17; A61K 40/11; A61K 40/31; A61P 35/00; C12N 5/0018; C12N 5/0638; C12N 9/226; C12N 15/111; C12N 15/87; C12N 2310/20; C12N 2501/2301; C12N 2501/2302; C12N 2501/2304; C12N 2501/2306; C12N 2501/2307; C12N 2501/231; C12N 2501/2312; C12N 2501/2315; C12N 2501/2318; C12N 2501/25; C12N 2501/51; C12N 2501/515; C12N 2510/00; C12N 2533/30; C12N 2533/50; C12N 2535/10

(57) ABSTRACT

Disclosed herein are micropatterned surfaces, particularly in the shape of micro-brushes having a base and a plurality of elongated elements. The micropatterned surfaces suitably biofunctionalized are particularly useful for activation and expansion of cells. Methods for activation and expansion of cytotoxic lymphocytes with decreased exhaustion potential are also provided, as well as means involving same to treat a patient in need thereof.

### FIELD OF THE INVENTION

The present disclosure relates to bio-functionalized patterned surfaces, e.g., bio-functionalized micro-brushes, e.g., for activation and proliferation (expansion) of lymphocytes, and to obtaining modified lymphocytic cell populations therewith. The present disclosure further relates to therapeutic uses of the modified lymphocytic cells populations.

### BACKGROUND OF THE INVENTION

Adoptive immunotherapy is a new, rapidly developing family of approaches for the treatment of cancer with autologous (patient's own) lymphocytes. Among other approaches, it includes immunotherapy with T cell modified to display chimeric antigen receptors (CAR). This approach is based on T cells modified with engineered receptors containing two main parts: (i) an antibody-based extracellular domain, specific to a cancer marker and (ii) an intracellular domain that generates activating and costimulatory signaling. The engineered T cells are then transfused back to the patient's body and attack the tumor. Several CAR T-cell immunotherapies were approved by the FDA, for example, for treatment of acute lymphoblastic leukemia (ALL), B-cell non-Hodgkin's lymphoma and multiple myeloma. An additional immunotherapy approach is based on tumor-infiltrating lymphocytes (TILs), which are isolated from the tumor stroma, assayed for neoantigen-specific recognition and expanded before being re-introduced to the patient. Additional adoptive immunotherapies employ T cells with engineered T cell receptors and natural killer (NK) cells. With more than 1000 ongoing or recruiting clinical trials, these methods appear as promising and revolutionary cancer therapies.

Despite encouraging developments in T cell-based immunotherapy, it still faces several challenges, with a prominent challenge relating to the ex vivo activation and expansion steps in the process of immunotherapeutic T cell production. The number of obtained T cells are understandably critical for obtaining therapeutically sufficient amount, and the initial amount of T cells is also important for the efficient transfection with CARs. Additionally, the over-stimulation of the isolated T cells may lead to exhaustion of the cells inter alia, via the expression of TIM3 and/or programmed death 1 (PD-1) receptor, decreasing significantly their ability to proliferate and act against cancer cells, as well as turning them vulnerable to inhibition by cancer or tumor microenvironment cells, even if the activated T cells may be still functional otherwise, e.g., in vitro.

In general, ex-vivo T cell activation (inter alia for adoptive immunotherapy) requires the following three stimuli: binding of T-cell receptor (TCR)-CD3 complex to a suitable recognition member that produces an activating signal, binding of costimulatory receptor CD28 to a suitable recognition member that produces a costimulatory signal, and supply of the cytokine interleukin-2 (IL-2), where the suitable recognition member (or stimulant) may be an antibody. In current clinical practice, the first two stimuli are routinely supplied by using magnetic beads (such as Thermo Fisher's Dynabeads™) coated with anti-CD3 and anti-CD28 antibodies. The third stimulation is usually achieved by using recombinant IL-2. The Dynabeads™ are then separated from the cells using a strong magnet.

However, these commercially available beads, which were initially designed for cell isolation, are not optimized for the ex vivo activation of T cells, and in particular fail to reproduce the physical features characterizing the interface between T cells and their targets in vivo. T cells contact target cells in vivo using microscale actin-rich protrusions called “microvilli”. The topology of microvilli does not only increase the T cell-target interface, but it is also assumed to play an important role in the spatial reorganization of signaling molecules that regulate the initial T cell response. These microvilli tips are enriched with T cell receptors (TCRs) and produce tight contacts with the target cell's membrane. The shape of the microvilli promotes the spatial exclusion of large phosphatase CD45 molecules from these contacts, which, in turn, shifts the kinase-phosphatase equilibrium in favor of the phosphorylation, resulting in enhanced TCR signaling. None of these features is present on the commercially available beads.

Moreover, prolonged incubation and use of increased bead concentration do not show any improvement in the activation or expansion of T cells. Furthermore, they may lead to nonspecific binding and increase the risk of cell trapping and damage as a result of the beads' shear forces.

Many micro/nano-scale platforms, such as liposomes, exosomes, biodegradable polymer particles, latex beads, and carbon nanotubes polymer composites have been proposed and are used as alternatives for T cell activation (Prakken, B. et al., Nat. Med. 2000, 6 (12), 1406-1410; Zitvogel, L. et al., Nat. Med. 1998, 4 (5), 594-600; Steenblock, E. R. et al. Mol. Ther. 2008, 16 (4), 765-772; Oosten, L. E. M. et al., Blood 2004, 104 (1), 224-226; Fadel, T. R. et al., Nat. Nanotechnol. 2014, 9 (8), 639-647). However, most studies of such artificial antigen-presenting cells (APC) systems have focused on chemical signals needed for activation and proliferation of specific T cells, without considering the physical requirements mentioned above. In addition, the reported platforms for replacing Dynabeads™ fail to match the need for scalability in their production process and exhibit disadvantages, such as their limited use for specific cell phenotype, expensive production, and they are generally ineffective for cell proliferation.

T cells and Natural Killer (NK) cells were recently stimulated on arrays of rigid ZnO-based or Si-based vertical nanowires with tunable elasticities (G. Le Saux, et al., Adv. Mater. 2019, 31; V. Bhingardive, et al., Small 2021, 17, 2007347′). It was shown that both the nanowire elasticity and nanowire-induced micro-topology produce distinct stimulatory effects on these cells (V. Bhingardive, et al., Nano Lett. 2021, 21, 4241; V. Bhingardive, et al., Adv. Funct. Mater. 2021, 31). Notably, elastic micro-pillars have been previously used for studying mechanical forces exerted by cells, as well as for the activation of helper T cells (W. Jin, et al., Proc. Natl. Acad. Sci. U.S.A. 2019, 116, 19835). Furthermore, micro-pillars coated with the natural pMHC ligands for T cell receptors were recently used to activate cytotoxic murine T cells (R. Basu, et al., Cell 2016, 165, 100). Furthermore, patent publication U.S. Pat. No. 10,301,590 relates (among others) to systems and methods for expansion of cells such as T cells based on an electrospun blended polydimethyl siloxane and polycaprolactone mesh substrate of specific porosity, and US patent application with publication number US20150030619 discusses T cells proliferation on polydimethyl siloxane slabs and beads of varying bulk mechanical properties.

Nevertheless, there remains an unmet need for an efficient, readily available, and scalable solution for ex-vivo T-cells activation and expansion, without impairing their activity or inducing exhaustion.

### SUMMARY OF THE INVENTION

Thus, in an aspect of the present disclosure provided herein a micropatterned surface comprising a base and a plurality of elongated elements disposed outwards essentially perpendicular to the base, the micropatterned surface consisting essentially of a soft elastomer polymer having a bulk elastic modulus of between 0.01 and 100 mega-Pascals, wherein the elongated elements are characterized by a bending stiffness of between 0.7 micro-Pascal*meter and 8 Pascals*meter, by a length of between 0.5 and 15 micrometers, by a thickness of between 0.25 and 3 micrometers, and further wherein the elongated elements are placed in a density of between 25,000 and 4,000,000 per square millimeter. Optionally, the bulk elastic modulus is between 0.1 and 5 mega-Pascals. Optionally, the bending stiffness is between 7 micro-Pascal*meter and 450 milli-Pascals*meter. Optionally, the length of the elongated elements is between 1.5 and 5 micrometers. Optionally, the density of the elongated elements is between 50,000 and 275,000 micro-pillars per square millimeter. Optionally, the soft elastomer is a poly(dimethyl siloxane), a poly(acrylamide), or a thermoplastic elastomer. Optionally, the micropatterned surface is coated with a linking moiety. Optionally, the linking moiety is a bifunctional molecule comprising a first binding domain and a second binding domain, wherein the first binding domain bound to the surface of the polymer, and the second binding domain being capable of binding a biologically active moiety. Particularly, the linking moiety may be 3-(aminopropyl)-triethoxysilane or thiol-polyethylene glycol-biotin. Optionally, the micropatterned surface may be functionalized with a biologically active moiety, wherein the biologically active moiety is a ligand to a molecule expressed on the surface of a living cell. Optionally, the living cell is a cytotoxic lymphocyte, such as T cell selected from the group consisting of CD8⁺ T cells, CD4⁺ T cells, γδ-T cells, and natural killer T cells. Optionally, the biologically active moiety is characterized by binding to and activating and/or stimulating at least one of the T cell receptor (TCR)-CD3 complex and/or the CD28 molecule on the surface of the living cell, such as an anti-CD3 antibody and/or an anti-CD28 antibody. Optionally, the micropatterned surface is further functionalized with an auxiliary moiety, such as an albumin. Optionally, the soft elastomer is poly(dimethyl siloxane) comprising between 2 and 25% of cross-linking moiety. Optionally, the surface is sterile.

In a further aspect provided herein a method of manufacturing of a micropatterned surface as defined above, the method comprising providing a positive mold of the micropatterned surface, wherein the material of the positive mold is silicon or a metal, coating the positive mold with an anti-adhesive hydrophobic layer, further coating the coated positive mold with a thermoset polymer, curing the thermoset polymer on the positive mold to obtain a negative mold, coating the negative mold with an anti-adhesive hydrophobic layer, and further filling the coated negative mold with a soft elastomer or soft elastomer precursor mixture. Optionally, the method is wherein the soft elastomer is poly(dimethyl siloxane) comprising between 2 and 25% of cross-linking moiety. Optionally, the method further comprises coating the surface with a linking moiety. Particularly, the coating may comprise contacting sequentially the surface with an oxidizing agent, and with a siloxane-containing linking moiety. Optionally, the oxidizing agent is oxygen plasma, and the siloxane-containing linking moiety is 3-(aminopropyl)-triethoxysilane. Optionally, the method further comprises combining the surface with a biologically active moiety characterized by binding to and activating and/or stimulating at least one of the T cell receptor (TCR)-CD3 complex and/or the CD28 molecule on the surface of a living cell, such as an anti-CD3 antibody and/or an anti-CD28 antibody. Optionally, the method further comprises coating the surface with an albumin. Optionally, the method further comprises sterilizing the surface.

In a further aspect provided herein a method for expanding cytotoxic lymphocytes, the method comprising: (a) providing a cell collective comprising cytotoxic lymphocytes isolated from a biological sample obtained from a donor; (b) contacting the cell collective isolated in step (a) with a micropatterned surface as defined in any one of claims 1-14, to effect the activation of the cytotoxic lymphocytes in the cell collective; (c) recovering the cell collective obtained in step (b) comprising activated cytotoxic lymphocytes from the micropatterned surface, and (d) culturing the collective, in the presence of a cytokine, thereby obtaining an expanded population of cytotoxic lymphocytes in the cells collective. Optionally, the method further comprises isolating the cytotoxic lymphocytes from the cells collective. Optionally, the at least one cytokine is selected from the group consisting of IL-1, IL-2, IL-4, IL-6, IL-7, IL-10, IL-12, IL-15, IL-18, and TNF. Optionally, the contacting (b) is performed in presence of at least one cytokine, which may be same or different from the cytokine used in step (d). Optionally, the contacting is performed for a time period of between 15 minutes and 72 hours, such as for example for a time period of between 2 and 8 hours. Optionally, the culturing of the cytotoxic lymphocytes is carried out for a time period of between about 3 days to about 12 days, such as between about 4 days and about 7 days. Optionally, as T cell are CD8⁺ T cells, CD4⁺ T cells, γδ-T cells, or natural killer T cells. Optionally, the method is characterized in that the micropatterned surface comprises a ligand that is characterized by binding to and activating and/or stimulating at least one of the T cell receptor (TCR)-CD3 complex and/or the CD28 molecule on the surface of the cytotoxic lymphocytes, such as an anti-CD3 antibody and/or an anti-CD28 antibody. Optionally, the method further comprises genetically modifying the cytotoxic lymphocytes to express a chimeric antigen receptor. Optionally, the method further comprises enriching the cytotoxic lymphocytes for an antigen-specific cell population. Optionally, the enriching for T cells is performed after the isolation from the biological sample but prior to the contacting with the micropatterned surface. Optionally, the micropatterned surface is functionalized with a first ligand characterized by binding to and activating the TCR-CD3 complex and a second ligand characterized by binding to and activating the CD28 molecule on the surface of the cytotoxic lymphocyte, the contacting is performed for a time interval of between 20 and 28 hours, wherein the cytokine of step (d) is IL-2, and wherein the culturing is for a time interval of between 4 days and 7 days. Optionally, method further comprises genetically modifying the cells collective comprising cytotoxic lymphocytes isolated in step (a) to express a chimeric antigen receptor (CAR). Optionally, the genetically modifying comprises any one of steps of electroporating the cells in presence of a Cas9 protein bearing CAR genetic material or in presence of double-stranded DNA encoding for CRISPR-Cas9 enzymatic system bearing the CAR genetic material, or transfecting the cells with a suitable viral carrier loaded with genetic material encoding the CRISPR-Cas9 and the CAR. Optionally, the cytotoxic lymphocytes are tumor infiltrating lymphocytes isolated from a tumor biopsy obtained from a cancer patient in need of treatment.

In a further aspect provided herein a method of treating or preventing a disease in a subject in need thereof comprising administering to said subject an effective amount cytotoxic lymphocytes obtained by using a micropatterned surface as described herein or obtainable by the method as defined herein, wherein said cytotoxic lymphocytes are derived from said subject, and wherein said disease is a cancer, an inflammatory disease, or an infection. Optionally, the expanded population of cytotoxic lymphocytes comprise CAR T cells. Optionally, the expanded population of cytotoxic lymphocytes comprise tumor infiltrating lymphocytes.

In a further aspect provided herein a kit for practicing the method for expanding cytotoxic lymphocytes and/or for treating or preventing a disease in a subject in need thereof, the kit comprising a micropatterned surface as defined herein, and instructions for using same. Optionally, soft elastomer of the micropatterned structure is poly(dimethyl siloxane) comprising between 2 and 25% of cross-linking moiety, wherein the kit further comprises a solution in ethanol or a weighed quantity for reconstitution in ethanol of 3-aminopropyl-triethoxysilane in a suitable container, and wherein the instructions contain directions to expose the micropatterned surface wherein the soft elastomer is poly(dimethyl siloxane) comprising between 2 and 25% of cross-linking moiety to an oxidizing agent and to the 3-aminopropyl-triethoxysilane solution. Optionally, the kit further comprises a solution or a weighed quantity for reconstitution of anti-CD3 antibody, preferably in a biologically acceptable medium, and/or a solution or a weighed quantity for reconstitution of anti-CD28 antibody, preferably in a biologically acceptable medium. Optionally, the kit further comprises a solution of or a weighed quantity for reconstitution of IL-2, preferably in a biologically acceptable medium. Optionally, all components of the kit, optionally excluding the instructions, are sterile.

### BRIEF DESCRIPTION OF THE DRAWINGS

Embodiments will now be described, by way of non-limiting example only, with reference to the accompanying drawings, in which:

FIG. 1. (a) Schematic illustration of activation and proliferation of T cells on the antibody-functionalized micro-brushes. (b) Pseudo colored SEM micrograph of a T cell stimulated on the micro-brush.

FIG. 2. (a) z-stack and (b) top view confocal images of CD8⁺ T cells activated on short the micro-brush surfaces. The cells were stained with Alexa Fluor 555 phalloidin to visualize the cytoskeleton, DAPI for nuclei, and APC-labeled anti-CD107a as a marker of degranulation (c) Average amount of CD107a per cell vs. type of activating surface. (d) Amount of IFN-γ vs. type of activating surface. The analysis was performed with ANOVA followed by Tukey's multiple-comparison tests using the GraphPad Prism software. *p<0.05, **p<0.01, ***p<0.001, ****p<0.0001, ns: not significant. All scale bars are 5 microns.

FIG. 3. Confocal images of T cells showing phospho ZAP-70, after stimulation on (a) short micro-brushes and (b) long micro brushes. Cells were stained with phalloidin for cytoskeleton (red) and DAPI (blue) for nuclei and phospho ZAP-70 (green). (c) Quantification of phospho ZAP-70 in T cell on different PDMS surfaces and controls. The analysis was performed with ANOVA followed by Tukey's multiple-comparison tests using the GraphPad Prism software. *p<0.05, **p<0.01, ***p<0.001, ****p<0.0001. Combination of brightfield view of T cells stimulated on (d-e) short micro-brushes and on (f-g) long micro-brushes to view co-localization of ZAP-70. All scale bars are 5 μm.

FIG. 4. Mechanical interaction of human CD8⁺ T cells with different brushes. (a) 3D and (b) cross section z-stack images of T cells on the short and long micro-brushes. Cells were stained in green for membrane (CellMask) and in blue for the nucleus (DAPI). To visualize the substrate topology, both surface bound mouse anti-human CD3 and CD28 were labelled with anti-mouse Alexa 647. (c) and (d) pseudo colored SEM images of T cells on the short and long micro-brushes, respectively. (e) Average projected cell area on vs. type of stimulating surface. The analysis was performed with ANOVA followed by Tukey's multiple-comparison tests using the GraphPad Prism software. *p<0.05, **p<0.01, ***p<0.001, ****p<0.0001. All scale bars are 5 μm.

FIG. 5. (a) Proliferation of human CD8⁺ T cells on micro-brushes and control surfaces (cells/ml versus number of days). (b) Population doubling of human CD8⁺ T cells on micro-brushes and control surfaces over time (number of days).

FIG. 6. Feature and time-point specific ranking of T-cell activation surfaces for the feature: (a) proliferation on day 3, (b) proliferation day 7.

FIG. 7. Feature ranking of T-cell activation surfaces for the time point Day 1 and the feature: (a) activation marker CD69, (b) activation marker CD107a.

FIG. 8. Feature and time-point specific ranking of T-cell activation surfaces for the feature: (a) short-term exhaustion marker TIM3 measured on Day 3, (b) long-term exhaustion marker TIM3 measured on Day 3, (c) short-term exhaustion marker TIM3 measured on Day 7, (d) long-term exhaustion marker TIM3 measured on Day 7.

FIG. 9. Feature and time-point specific ranking of T-cell activation surfaces for the feature: (a) transfection marker determined on day 3, (b) transfection marker determined day 7.

FIG. 10. Global combined ranking of T-cell activation surfaces for the collective of measured features.

FIG. 11. Viability and transfection efficiency of T cells activated on various surfaces 24 hours after electroporation: (a) viability of cells at dsDNA transfection, (b) viability of cells for Cas9 transfection, (c) transfection efficiency viability of cells at dsDNA transfection, and (d) transfection efficiency viability of cells at Cas9 transfection.

FIG. 12. Efficacy of CAR T cells against JIMT1 cell line.

### DETAILED DESCRIPTION OF THE INVENTION

The present disclosure is generally drawn to micropatterned surfaces, particularly to micro-scale pillar-structured elastomers; to the fabrication of these micropatterned surfaces, that are functionalized or functionalizable with functionalizing moieties, e.g., an activating and a costimulatory biological ligands (i.e., bio-functionalized); to methods of activation and expansion of immune-therapeutic cells, such as cytotoxic lymphocytic cells; to methods of using thus activated and expanded cells in treatment of cancer in subjects in need thereof; to devices comprising said micropatterned surfaces; to the micropatterned surfaces and devices for use in activation and expansion of immune-therapeutic cells and in methods of using thus activated and expanded cells; and to kits comprising a device or an activation surface as described herein and complementary reagents for bio-functionalization and/or culturing lymphocytic cells.

It has been unexpectedly found that the bio-functionalized micro-structured elastomers described herein that are shaped as micro-brushes whose surfaces are generally characterized by controlled three-dimensional microstructure, biocompatibility, mechanical stiffness, and coating with activating molecules (i.e., the surfaces are bio-functionalized with what is referred to herein as “ligands”), may selectively affect the activation, expansion, exhaustion, and desired cell types. Without being bound by a specific theory, it is currently believed that the surface of the micropatterned surfaces, e.g., micro-pillar array, provides an artificial microenvironment mimicking the surface of living cells (e.g., cancer cells or antigen-presenting cells) and by contacting e.g., lymphocytes, such as human T cells or peripheral blood mononuclear cells (PBMC), the activation and expansion of the population thereof may be performed to advantageous results unattainable with the currently used means, such as Dynabeads™ magnetic functionalizable beads, as elaborated below.

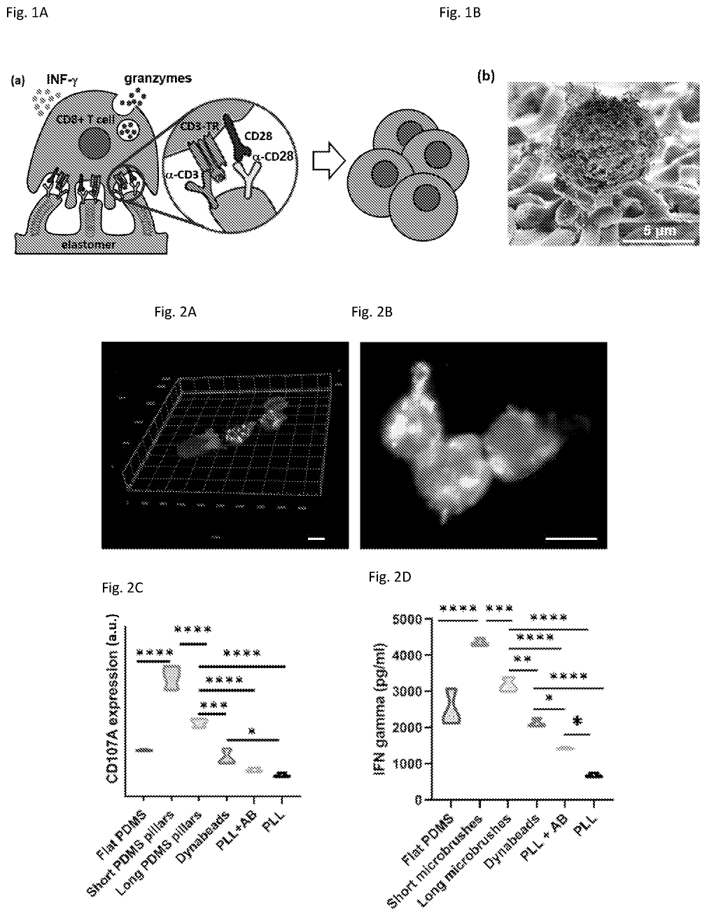

As demonstrated in the appended examples, the micropatterned activation surfaces, such as micro-pillar array, activate and facilitate expansion of contacting cytotoxic lymphocytes in a rapid and efficient manner, with minimal exhaustion effects. To demonstrate the principle, a section of the micro-pillars array, functionalized with activating and costimulatory ligands (antibodies), in contact with a CD8⁺ T cell, is schematically shown in FIG. 1A. This figure schematically demonstrates interaction between a CD8⁺ T cell and a micro-pillar array (indicated by “elastomer” in the bottom assembly), with the contact between a micro-pillar and the cell enlarged in the circular inset, along with the contact between the T-cell receptor-CD3 complex (denoted by “CD3-TR”) and its corresponding antibody on the micro-pillar (denoted “α-CD3”), and the contact between CD-28 and its corresponding antibody (denoted as “CD28” and “α-CD28”, respectively). On the left side of the scheme, cell excretion of interferon gamma (“INF-γ”) and granzymes is demonstrated as plurality of dots outside the cell and within an internal circle. The effect of the contact is demonstrated by the right-headed arrow towards the multiplication of the cell. The number of proliferated cells is shown for demonstration only and does not necessarily imply 1 to 4 expansion.

In particular, and as shown in the Examples section below, it has been found by the inventors that the micro-brush (i.e., micro-array) topology greatly enhanced the magnitude of degranulation and cytotoxicity of T cells, as revealed by both CD107a expression and INF-γ release, when compared to those obtained by contacting the cells with a flat surface with the same elasticity and functional ligand coating, or with commercially available functionalized magnetic beads, and also vis-à-vis uncoated flat surfaces with the same mechanical properties. As currently believed and has been found by the inventors, T cell activation may depends on the micropattern topology, with the highest activation produced by brushes with relatively short, standing micro-pillars that comply to the forces exerted by T cells, and allow the protrusion of the cell membrane into the areas between the pillars (as shown in FIG. 1B, where the scale bar is 5 microns, denoted as “5 μm”). The length of brush villi is shown for demonstrated example only and does not necessarily imply the optimal or desired length.

Surprisingly, it is presently demonstrated that the micro-array-induced topology dictated by pillar length, did not affect early signaling in the stimulated T cells, suggesting, without being bound to any theory that the mechanism of topologically induced activation unlikely involves any modulation of TCR triggering. Finally, it has been found that the micro-brush (micro-array) induced activation led to the enhancement in T cell proliferation (expansion), both in terms of proliferation magnitude and duration, with the short micro-pillars demonstrating the highest effect. Without being bound to theory, these findings suggest that the CD8⁺ T cells tested, sense both the surface topology (i.e., the micro-pillar arrangement) and elasticity (or “resistance” of the micro-pillars) and that an optimized micro-brush/array is an alternative to the currently available platforms for therapeutic ex vivo activation and proliferation of T cells.

As further demonstrated in the examples below, the PBMC cells, including the T cells, activated on the micropatterned surfaces as described herein, were not only activated to no less extent that the currently used industry standard, but supported proliferation to significantly higher numbers, culturing to higher expansion, and were characterized by significantly less exhaustion, as demonstrated by lower prevalence of emergence of programmed death receptor 1, the prominent feature of cancer armamentarium to combat immune system.

Thus, in one aspect provided herein micropatterned surfaces. These micropatterned surfaces may be regarded as “bioactive”, for example, “activating” or, alternatively, “downregulating”, depending on the nature of the ligands used for bio-functionalization, as described below. The micropatterned surfaces are thus usually functionalized or functionalizable with biological ligands, as defined herein below, to convey specific biological signals to a variety of cells. Particularly, as elaborated below, and currently preferably, the signals are biochemical signals to effect and enhance the activation and proliferation of the T cells. The micropatterned surface may thus bear functionalizing moieties (e.g., “ligands”), physically or chemically attached to the surface of said micropatterned surface. Specifically, the functionalizing moieties, as described in greater detail below, are targeting T cells, and may therefore include an activating moiety, a costimulatory moiety, and/or a combination of these. The micropatterned surface may thus be referred to as “bio-functionalized”, or “functionalized”, which are used herein interchangeably unless the context clearly dictates otherwise.

The pattern of the micropatterned surface, as described herein, may be presented in form of a plurality of micro-scale elongated elements, e.g., pillars or villi, protruding from the surface. Therefore, the micropatterned surface comprises a base surface (or simply a base) and a plurality of elongated elements extending distally outwards from the base surface, in an essentially perpendicular manner as defined herein. Generally, the elements may be same or different, and may be ordered randomly or in a predetermined pattern. The micropatterned surface may therefore be referred to as “micro-brush”. Currently preferably, the elongated elements may be presented in a form of prismatic or cylindrical micro-pillars. Therefore, the term “micro-brush” in reference to the micropatterned surfaces may be interchangeably referred to herein as “micro-pillars”, “micropillars”, “micro-pillar array”, and the like, unless the context clearly dictates otherwise. The micro-brushes contain a plurality of elongated protrusions, e.g., cylindrical pillars. The micro-pillars may be distributed randomly, or in a structured manner.

For example, the micro-pillars may be arranged at a density of about 25,000 to about 4,000,000 micro-pillars per square millimeter, which correspond to approximate pitch values of 6 microns and 0.5 microns, respectively, in ordered symmetrical micro-brushes. In currently preferred embodiments, the density may be between 50,000 and 275,000 micro-pillars per square millimeter, corresponding to approximate pitch values of about 4 and 2 microns, respectively, in ordered symmetrical micro-brushes. Currently preferably, the elongated elements on the micro-brushes are essentially evenly distributed, with the pitch, i.e., with a periodicity of element placement, of between about 0.5 μm to about 10 μm, e.g., about 1, 2, 3, 4, 5, 6, 7, 8, or about 9 μm. Currently preferably, the pitch is essentially similar in both planar axes of the micro-brush, and is between 2 and 5 μm.

The diameter of the micro-pillars, i.e., the maximal thickness of the elongated elements at base, may be between about 0.2 μm to about 4 μm, e.g., between about 0.3 μm to about 2 μm, e.g. about 0.5 μm, or about 0.6 μm, or about 0.7 μm, or about 0.8 μm, or about 0.9 μm, or about 1 μm, or about 1.2 μm, or about 1.5 μm, or about 2 μm, or in a range of between these values. Currently preferably, the diameter of the elongated elements is between 0.2 μm and 3.5 μm, e.g., between 0.25 and 3 μm, or between 0.5 and 1.5 μm, or between 1.5 and 2.5 μm, or between 0.7 and 2.5 μm.

Preferably, the pitch and the diameter may be adjusted such that the distance between the elongated elements' adjacent surfaces may be between 1 and 4 μm.

The length of the elongated elements, e.g., micro-pillars, may be from about 0.5 and about 15 μm, e.g., between about 1 μm to about 10 μm (e.g., about 2.5 μm to about 7.5 μm, or about 3 μm to about 6 μm), currently preferably between 1.5 and 5 μm. Thus, for example, in some of the appended examples the micro-pillars with length of about 5 μm or about 10 μm also referred to as “short” or “long” micro-brushes/arrays, respectively. In particular embodiments, the elongated elements are in form of micro-pillars with the diameter of between 0.25 and 3 micrometers and the length of between 1.5 and 5 micrometers. In further specific embodiments, the elongated elements of the present disclosure have (each) a length of about or below 5 μm, e.g., between 1 and 5 μm, and a diameter of between 0.3 and 1.3 μm, e.g., about 0.5 μm, or about 1 μm. In some further specific embodiments, the elongated elements of the present disclosure have (each) a length of between 1 and 2 μm, and a diameter of between 0.3 and 0.7 μm, e.g., about 0.5 μm, or a length of between 3 and 7 μm, and a diameter of between 1.5 and 2.5 μm, e.g., about 2 μm.

The micropatterned surface may be formed on any suitable material, but in particularly preferred embodiments, the micropatterned surface is formed on or of a soft elastomer, as elaborated in greater detail below. In currently preferred embodiments the micropatterned surface consists essentially of a soft elastomer. In a broadest aspect, the elastic modulus of the elastomer may be between 0.01 MPa and 100 MPa, preferably between 0.1 MPa and 5 MPa. In some specific embodiments, the elastic modulus of the soft elastomer as described herein is below 1.5 MPa, further preferably below 1 MPa, particularly below 0.9 MPa, below 0.8 MPa, below 0.7 MPa, below 0.6 MPa. Thus, the bulk elastic modulus of the soft elastomer may preferably be between 0.01 MPa or 0.1 MPa, and 1.5 MPa, or between 0.01 MPa or 0.1 MPa and 1.2, or 1, or 0.9, or 0.8, or 0.7, or 0.5, or 0.5 MPa.

In some embodiments, the polymer suitable for preparing the micro-brush of the present disclosure is a cross-linkable, e.g., thermoset, elastomer polymer, such that the composition may be suitable for adjusting the mechanical properties of the pillars to the desired strength, e.g., desired mechanical properties as set forth herein. In some preferred embodiments, the polymer is poly-dimethyl-siloxane (PDMS). In some alternative embodiments, the polymer is a polyacrylamide or polyacrylamide gel. In some further embodiments, the polymer is a thermoplastic elastomer. The thermoplastic elastomers may be selected from styrene-butadiene-styrene block copolymer, styrene-ethylene-butylene-styrene block copolymer, polyurethanes, and other thermoplastic elastomers known in the art, as defined, e.g., in ISO 18064. In the embodiments wherein the polymer is cross-linkable, the ratio between the monomer/pre-polymer and the cross-linking moiety (also referred to interchangeably herein as a “hardener”, “curing moiety”, and the like) may be adjusted according to the desired mechanical properties, such as the elastic modulus of the material, or the bending stiffness of the protruding elements. As demonstrated in the examples' section below, the elastic modulus of the PDMS material used, produced major effects on the performance of the micropatterned surfaces.

The soft elastomer suitable for preparing the micro-pillar array of the present disclosure is usually such that the resulting elongated elements, e.g., micro-pillars, allow direct contact and interaction between the cells to the element distal parts, i.e., tips, such that, for example, a polymer the use thereof allows obtaining deformable elongated elements characterized by bending stiffness of between about 1 μPa*m to 10 Pa*m (i.e., from 1 micro-Pascal*meter to 5 Pascal*meter), e.g., between 7 μPa*m and 8 Pa*m. As known to a skilled person in the field of the invention, the bending stiffness is a standard for measuring deformability, which is deflection in case of a cantilever, and is determined as the force applied on the cantilever edge perpendicularly to the cantilever divided by the resulted deflection. Thus, in reference to the micro-pillars, the bending stiffness as defined herein is an expression of the force that is required to deflect the micro-pillar tips from the respective micro-pillar axes, which are preferably essentially perpendicular to the support whereon they are disposed. In particular embodiments, the micro-pillar of the present disclosure is characterized by a bending stiffness in the range of between about 7 μPa*m and 450 mPa*m, e.g., between about 7.3 μPa*m and 430 mPa*m, which corresponds approximately to the bending stiffness of elongated elements of 0.5 μm in diameter and 10 μm in length, and of 3 micrometers in diameter and 2 micrometers in length. In particular further embodiments, the bending stiffness may be in an exemplary range of between 30 μPa*m to about 20 mPa*m, such as from about 0.1 mPa*m to about 10 mPa*m. In further particular embodiments the micro-pillar of the present disclosure is characterized by a bending stiffness of between about 0.3 mPa*m to about 2 mPa*m, typical to the “long” and “short” PDMS pillars prepared as described in the Examples 1-4 below. In further preferred embodiments, the bending stiffness is about 2 mPa*m, typical to the short PDMS pillars prepared as described below. Particularly preferably, the bending stiffness of the soft elastomer elongated elements may be between 0.03 mPa*m and 10 mPa*m, e.g., between 0.05 and 2 mPa*m. The terms “bending stiffness” and “flexural stiffness” are used herein interchangeably.

As mentioned, the elongated elements are preferably disposed on the substrate in an essentially perpendicular manner, which should be construed herein as the axis of the micro-pillar forming essentially a right angle with the support, i.e. about 90°. In a broader sense, the “essentially perpendicular” micro-pillars may have their axes forming angles with the support of a value between 75° and 105°. The terms perpendicular and essentially perpendicular are therefore used herein interchangeably.

Thus, in particular embodiments, provided herein a micropatterned surface comprising a base and a plurality of elongated elements disposed outwards essentially perpendicular to said base. The micropatterned surface preferably consists essentially of a soft elastomer polymer having a bulk elastic modulus of between 0.1 and 5 MPa. The elongated elements are preferably characterized by a bending stiffness of between 7 μPa*m and 450 mPa*m. Further, the elongated elements are preferably characterized by a length of between 1.5 and 5 micrometers. Further, the elongated elements are preferably characterized by a thickness of between 0.25 and 3 micrometers. Further preferably, the elongated elements are placed in a density of between 50,000 and 300,000 per square millimeter. The particularly preferable soft elastomer is PDMS or polyacrylamide.

In particular embodiments, wherein the polymer is PDMS, the cross-linking moiety may be a bifunctional or higher valency moiety that is able to react with PDMS monomers or pre-polymers. For PDMS, there are currently several ready-to-use kits available on the market, inter alia, the ones produced by the Dow™ company, under the trade name Sylgard™. These kits contain the base and the hardener, whereas the base contains dimethyl siloxane monomers or oligomers, some of them being optionally vinyl-terminated, and the hardener comprises the crosslinking moiety. In some embodiments, particularly wherein said polymer is PDMS obtainable from Sylgard™ 184 silicone elastomer kit, the ratio between the base and the hardener may be between about 50:1 to about 4:1, of polymer to hardener. Currently preferably, the soft elastomer is poly(dimethyl siloxane) comprising between 2 and 25% of a cross-linking moiety. In further specific embodiments, the ratio between the polymer and the hardener may be 40:1, 35:1, 30:1, 25:1, 20:1, 18:1, 15:1, 12:1, 10:1, 7:1, or 5:1, or any value therebetween. Thus, using a higher amount of hardener it may be possible to obtain harder surfaces, e.g., characterized by a higher bulk elastic modulus and/or the bending stiffness of the elongated elements. In some embodiments, wherein the polymer is polyacrylamide or polyacrylamide gel, the monomer is preferably selected from the group consisting of acrylamide and its derivatives, and the cross-linking moiety is preferably N,N′-methylenebisacrylamide.

The micropatterned surfaces, e.g., the presently disclosed micro-pillar arrays, may thus be functionalized with a biologically active moiety, e.g., with a molecule that specifically binds and conveys a biochemical signal to the adjacent cell. Thus, the micropatterned surfaces as described herein are functionalizable with at least one biologically active moiety. The biologically active moiety may therefore be a ligand of a molecule expressed on a surface of a living cell, e.g., the target cells. In currently preferred embodiments, the target cell is a lymphocyte, e.g., a cytotoxic lymphocyte. Preferably, the living cell is a T cell selected from the group consisting of CD8⁺ T cells, CD4⁺ T cells, γδ-T cells, and natural killer T cells.

By the term “ligand”, therefore, and also terms “stimulatory ligand”, “co-stimulatory ligand” and the like it is referred herein interchangeably to a member that upon binding to cells, e.g., lymphocytes, effects a change in the bound cell's homeostasis, e.g., activates the lymphocytes (directly or indirectly). Currently preferably, the “ligand” as herein defined binds to at least one receptor on the lymphocytes, e.g., the (TCR)-CD3 complex, e.g., “stimulatory ligand”, and/or the receptor CD28, e.g., “costimulatory ligand”, thereby triggering activation and expansion of the affected lymphocytes. Other stimulatory, co-stimulatory, and targeting molecules may be used, depending on the final application of the micropatterned surfaces. Additionally, the functionalized (or “bio-functionalized”, as used herein interchangeably) surfaces may contain further auxiliary moieties, e.g., to enhance the biocompatibility, to decrease the non-specific binding, etc. one prominent auxiliary molecule, is albumin. Therefore, the micropatterned surface is further characterized in that that the biologically active moiety is characterized by binding to and activating and/or stimulating at least one of the T cell receptor (TCR)-CD3 complex and/or the CD28 molecule on the surface of the living cell. In some embodiments, the ligand is an antibody directed against the TCR-CD3 complex and/or CD28, or any section thereof, such as an anti-CD3 antibody and an anti-CD28 antibody.

In various embodiments the concentration of the biologically active moiety (the “ligand” as used herein) as herein defined in the micro-pillar array according to the present disclosure may be between 0.001-0.1 fmol/mm², preferably between 0.01-0.7 fmol/mm².

The ligands and the auxiliary molecules may be directly adsorbed physically onto the micropatterned surfaces. However, in preferred embodiments, the bio-functionalizing moieties are adsorbed or chemically/biochemically bound to the micropatterned surfaces via a linking moiety. The linking moiety is preferably a bifunctional molecule comprising a first binding domain and a second binding domain, such that the first binding domain binds or is bound in use to the surface of polymer, and the second binding domain is being capable of binding the biologically active moiety. For example, when the polymer is PDMS-based surface, the linking moiety (e.g., 3-aminopropyl)-triethoxysilane, APTES, as described inter alia below) may be applied, after a suitable treatment of the surface, as elaborated below. The first binding domain is the silane moiety, whereas the second domain is the amine moiety. Other linking moieties suitable for some surfaces may include thiol compounds, biotin-avidin compounds, and others. For example, thiol-PEGₓ-biotin, wherein x is the number of repeating units of polyethylene glycol, may be used as a thiol and biotinated compound, with thiol being the first domain binding to the surface, and the biotin is bindable to a ligand carrying an avidin complementary part to biotin. Other linking moieties may be used as well known in the art.

Thus, in preferred embodiments, the micro-pillars of the present disclosure are functionalized with at least one ligand by at least one linking moiety (which may be monomeric, oligomeric or polymeric in nature, etc.). In further specific embodiments, the linking moiety is a siloxy residue, preferably a product of sequential reaction of oxidatively treated polydimethyl siloxane surface with (3-aminopropyl)-triethoxysilane, and with an antibody solution. Other linking moieties may also be used as known in the art.

Additionally, the micropatterned surfaces may comprise further functionalizing moieties, e.g., the auxiliary moieties. These auxiliary moieties may serve various functions when the micropatterned surfaces are in use. For example, the auxiliary moiety may block non-specific binding that is rather common in biological systems. Therefore the micropatterned surface as described herein may further be functionalized with an auxiliary moiety. Preferably, the auxiliary moiety is albumin.

Additionally, as the micropatterned surfaces may be primarily used in the field of biology and medicine, it is preferable that the micropatterned surfaces as described herein are sterile.

The micropatterned surfaces as described herein, may be fabricated or manufactured by a variety of techniques as known in the art.

For example, in a specific embodiment, the micro-pillar array may be prepared as generally described by B. Pokroy, et al. Science 2009, 323, 237, which is incorporated herein by reference. Briefly, silicon master molds, containing micro-brushes having a plurality of pillars may be fabricated using electron beam lithography and Bosch process. The positive molds contain multiple elongated elements, as generally described above regarding the thickness, pitch, arrangement, the length of the elements, and the angle versus the base. The silicon master molds may then be first coated with an anti-adhesive hydrophobic layer (e.g., trichloro (1H,1H,2H,2H-perfluorooctyl)-silane, as exemplified below, for a suitable time interval, e.g., overnight, in vacuum), to obtain an anti-adhesive hydrophobic layer on the silicon micro-pillars. Then, the coated silicon master molds may be further used to produce a negative mold. For this purpose, the positive master molds may be coated with a thermoset polymer/hardener mixture, e.g., as described above. Particularly, a mixture containing polydimethyl siloxane with polyfunctionalized siloxanes (as described below) or acrylamide gel precursor with a suitable bifunctional moiety may be used. The mixture is poured onto the surface of the positive silicon master mold, degassed and then cured for a suitable time as dictated by the polymer, e.g., for between 30 minutes to 48 hours, specifically for at least an hour, at a suitable temperature, between 20° C. and 120° C., specifically at about 60° C. The cured polymer may then be peeled off from the silicon master mold to obtain the negative mold, which in turn may be coated with a further anti-adhesive coating overnight, in vacuum (e.g., of trichloro (1H, 1H,2H,2H-perfluorooctyl) silane), to obtain a coated negative mold. The resulting coated negative polymer-based mold containing cavities of the depth, distribution, and diameter roughly corresponding to the desired length, pitch, and thickness of the micro-pillars, is next used for double replication by pouring a suitable polymer/hardener mixture, optionally thoroughly degassing, and curing at selected conditions (e.g., PDMS: hardener at a ratio of 10:1, as disclosed below, at 60° C., overnight). If excess polymer mixture is provided to fill-in the cavities of the mold, the excess polymer may be left to create a supporting surface, or may be removed prior to curing, and substituted for a different polymer to serve as a support. The cured micro-pillar polymer array may then be peeled off from the negative mold. The micro-pillar array of the present disclosure may be disposed (e.g., essentially perpendicularly) on a suitable support as previously described.

Thus, provided herein a method of manufacturing of a micropatterned surface as generally described herein and above, the method comprising providing a positive mold of the micropatterned surface, such that the mold is made of a suitable material, e.g., of silicon or a metal, e.g., nickel. The method preferably further comprises coating said positive mold with an anti-adhesive hydrophobic layer to furnish a coated positive mold. The method may further comprise coating the coated positive mold with a suitable polymer, e.g., a thermoset polymer, and curing the polymer on the coated positive mold, to obtain a negative mold. The method may further comprise coating the negative mold with an anti-adhesive hydrophobic layer to furnish the coated negative mold. The method may further comprise filling the coated negative mold with a soft elastomer or soft elastomer precursor mixture. Currently preferably, the filling of the coated negative mold is performed with poly(dimethyl siloxane) comprising between 2 and 25% of cross-linking moiety.

The negative mold may also be prepared using electrochemical deposition techniques, such as galvanoplasty.

Whereas the technique described above for the preparation of micro-pillar arrays provides for tunable properties of the micro-pillars and/or their support, the micro-pillar arrays may also be prepared by other techniques as generally known in the art, such as molding from a solid mold (e.g., as described by S. Ghassemi et al, PNAS, 2012, 109, 5328, incorporated herein by reference) with similar characterizing features of topology, elasticity, etc.

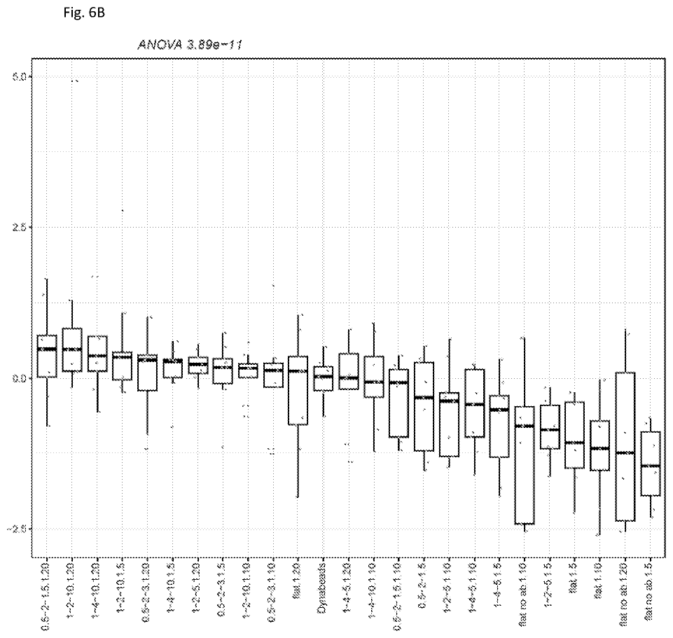

Some of the examples below may indicate that long pillars, e.g., having a length of about 10 μm, did not retain their free-standing shape, possibly due to the insufficient mechanical strength or handling during the experiment, but were rather bent over the surface. This may have resulted in some of the differences between the effect of the short and long micro-brushes on the contacting cells. The first difference was that the short micro-brushes as used in the Examples 1-4, namely having a length of about 5 μm, produced a pronounced topology at the cell-brush interface. This topology allowed the cells to protrude into the spaces between the pillars and restricted the cell-surface contact to the top 1-2 microns of the pillar area. On the contrary, the long micro-brushes (containing pillars having a length of 10 μm) did not produce a strong, membrane-invaginating topology and thus did not restrict the cell contact to the pillar tips. SEM images clearly revealed that the cells produced a relatively small contact area with the surface of the long pillars. The second difference between the short and long micro-brushes (micro-pillars) was that the short ones greatly complied with the centripetal forces applied by the cells, as clearly seen from the z-stack and SEM images (FIG. 4A-FIG. 4D). In this sense, a surface with standing brushes, where each of these acts as a tiny micro-spring that easily complies with the cellular forces, can be viewed as an “activating continuum” structure of many highly-elastic elements, and the overall effect of elasticity of this continuum is much stronger than that of a flat surface, or of a nearly flat surface as seen in SEM for long micro-brushes. The specific results described in the experiment do not necessarily imply that all elongated elements of a length above 5 μm would be expected to bend over. Moreover, the electron micrograph may be an artifact of the sample preparation process, whereas other microscopy definitively shows micro-brushes for the “long” pillars. Therefore, without being bound by theory it is suggested that the standing pillar topology may act as an artificial “enhancer” for the contact area between the cells and the stimulating surface (namely, the surface coated with the stimulatory ligands), contributing to an increased activation by the stimulatory antibodies and triggering increased cell activation.

Therefore, the polymer suitable for preparing the micro-pillar array of the present disclosure is preferably a soft elastomer, as described above. As described above, preferably the soft elastomer has a bulk modulus preferably between 0.1 and 5 mega-Pascals, and the elongated elements topology is selected such that their bending stiffness is 7 micro-Pascal*meter and 450 milli-Pascals*meter, e.g., between 0.03 mPa*m and 10 mPa*m

In some embodiments, as described herein, the polymer suitable for preparing the micro-array of the present disclosure is a thermoset polymer, such that the composition may be suitable for adjusting the mechanical properties of the pillars to the desired strength (e.g., to the properties set-out herein). In some preferred embodiments, the polymer is polydimethyl siloxane (PDMS). In some alternative embodiments, the polymer is a polyacrylamide or polyacrylamide gel. In the preferred embodiments, the soft elastomer polymer is a thermoset polymer, polymerizable on the mold, as described above. Further polymers include thermoplastic elastomers, as described above. Generally, the monomer or a pre-polymer, are combined together with the cross-linking moiety, and optionally an initiator. The mixture is then applied to the negative mold as described above, and optionally initiated, e.g., with a UV irradiation. Thermoplastic elastomers may be applied at elevated temperatures. The mold is left for final curing for the predetermined time interval, as know for each specific soft elastomer. As mentioned above, using a higher ratios of hardener to the monomer/pre-polymer, it may be possible to obtain harder surfaces, e.g., characterized by a higher bulk elastic modulus and/or the bending stiffness of the elongated elements.

As readily understood from the appended examples, the micro-pillar arrays are biocompatible, i.e., do not cause adverse effects to the cells, either by themselves or by chemicals eluting therefrom into the medium. The micro-pillar arrays may be biocompatible per se, for example being made of completely reacted thermoset polymer with known biocompatibility, such as PDMS, Teflon™, and others. Alternatively, the micro-pillar arrays may be made biocompatible by coating them with a suitable coating, e.g., with a protein, such as albumin.

Thus, the micropatterned surfaces as described herein, may be biofunctionalized, e.g., to improve biocompatibility, and/or to provide stimulatory machinery to effect the desired changes in the target cells, which when in use, interact with the micropatterned surfaces.

Therefore, in some embodiments, the ligand as herein defined is characterized by binding to and activating and/or stimulating at least one of the TCR-CD3 complex and/or the CD28 molecule on the surface of said cytotoxic lymphocytes (e.g., on T cells, for example on CD8⁺ T cells, e.g., human CD8⁺ T cells). In specific embodiments the ligand is an anti-CD3 antibody and/or an anti-CD28 antibody (i.e., an antibody directed against CD3 and/or CD28). In particular embodiments, the micro-pillar array as herein defined is functionalized with a first ligand characterized by binding to and activating the TCR-CD3 complex and a second ligand characterized by binding to and activating the CD28 molecule on the surface of said cytotoxic lymphocytes. In particular embodiments, the first ligand and the second ligand are antibodies directed to the TCR-CD3 complex and CD28 molecule on the surface of the cells being activated, respectively. Thus, the method of manufacturing micropatterned surfaces as described herein may further comprise combining the surface with a biologically active moiety characterized by binding to and activating and/or stimulating at least one of the T cell receptor (TCR)-CD3 complex and/or the CD28 molecule on the surface of a living cell. Particularly, the method may comprise combining the surface with an anti-CD3 antibody and/or an anti-CD28 antibody, and may further comprise combining the surface with an albumin.

Functionalization of the presently-disclosed micro-pillar array (for example, by coating) may be performed by any method known in the art, depending on the nature of the polymer and ligands used, for example as described by P. V'kovski, et al. Elife 2019, 8, (incorporated herein by reference), which is suitable for functionalizing, among others, PDMS-based surfaces with polypeptides. In particular, when micro-arrays are fabricated as described above using PDMS and functionalized with a mixture of ligands that are polypeptides (e.g., antibodies directed to the TCR-CD3 complex and to CD28, respectively), the micro-pillar arrays may be first treated with oxygen plasma for a suitable time, e.g., about 3 to about 30 seconds (e.g., 5 seconds). The treated surfaces may then modified with a linker or linking moiety (e.g., 3-aminopropyl)-triethoxysilane, APTES) by immersing into an ethanolic APTES solution of a suitable concentration, e.g. between 5 and 15%, e.g., 10%, for sufficient time for the linking moiety to react with plasma-activated PDMS, for example, for 10 minutes. APTES-modified micropatterned surfaces may then be rinsed with ethanol, and fixated at elevated temperatures, e.g., for 10 min in an oven at 50-90° C., preferably at 60° C., as previously described (P. V'kovski, et al. Elife 2019, 8). The fixated APTES-modified micropatterned surfaces may be kept in ethanol until further use, or sterilized and packaged. Before use, the micro-pillar array samples may then be incubated with the ligands, e.g., overnight at 4° C. in a suitable concentration, e.g., 2 μg/ml solution. The solution may preferably contain a mixture of the antibodies' solutions (e.g., α-CD3 and α-CD28 at the individual concentration of 1 μg/ml, each) in sterile phosphate buffered saline (PBS), then rinsed three times and stored in sterile PBS until use.

Thus, the method of manufacturing micropatterned surfaces preferably further comprises coating the surface with a linking moiety. Further preferably, the coating comprises contacting sequentially the surface with an oxidizing agent, and with a siloxane-containing linking moiety. The oxidizing agent may be selected as suitable and know in the art, and dictated by the chemical structure of the elastomer and the linking moiety. Preferably, the oxidizing agent is an oxygen plasma. The contacting time of the surface with an oxidizing agent is therefore dictated by the same considerations. For example, when the oxidizing agent is an oxygen plasma and the soft elastomer is polydimethyl siloxane, the surface may be contacted for a time interval of between several seconds to several minutes, preferably, to 1 to 10 seconds. In these embodiments, a particularly preferred linking moiety is 3-(aminopropyl)-triethoxysilane. The method may further comprise fixating the linking moiety, e.g., by heating the micropatterned surface coated with the linking moiety, to an elevated temperature, as described above.

Alternatively, particularly wherein the polymer is a thermoplastic elastomer as described above, the micropatterned surface may be prepared by the conventional processes known for these polymers, e.g., by injection molding, and also extrusion, blow molding, or thermoforming.

Finally, the method of manufacturing micropatterned surfaces preferably further comprises sterilizing the surface.

As shown herein by the inventors, the increased activation of T cells on the antibody-functionalized micro-arrays led to their increased proliferation, and lower exhaustion, rendering the micropatterned surface such as the micro-array suitable for production of T cells for immunotherapeutic purposes. In recent years, there has been emerging research aiming to develop new nano- and microscale materials that can potentially replace magnetic beads (among other methods), in the process of ex vivo expansion of T cells. Notably, any agent or ligand added to the cell media during the steps of cell activation and proliferation currently used for production of T cells for immunotherapeutic purposes must be completely sorted out from the cells' media upon completion of the activation stage, which is a challenging step. Incomplete sorting can lead to the presence of residual activating agents in the cell culture, whose effect on the therapeutic function of cells in the patient's body is unclear. Additionally, administering uncaptured non-degradable residues of activating media, such as non-degradable beads, may lead to serious complications upon (re) administration to a patient in need thereof, including embolism. Currently disclosed is a different approach, by which cells are stimulated on a surface with topological and elastic features that provide the improved physical cues for cell activation and expansion, including ligands that are immobilized to the surface and are thus easily separated from the cells. Practically, upon activation, populations of cytotoxic cells are separated from the surface, thereby excluding residues of stimulating ligands therein. Furthermore, activation on the micro-pillar array surfaces of the invention facilitates exposure of the contacting cells to a reproducible amount of physical and chemical cues, resulting in homogeny in the cell populations obtained which is advantageous for effective immunotherapies.

Thus, by a further aspect the present disclosure provides a method for activating and expanding/proliferating of lymphocytes, by steps including contacting the lymphocytes with the functionalized micropatterned arrays as described herein, to obtain an activated population of cytotoxic lymphocytes, and subsequently culturing the activated population of cytotoxic lymphocytes under suitable conditions such that the cells are expanded and may be transfused back into their donor or any other suitable subject in need thereof.

Thus, generally, provided herein in some embodiments a method for expanding cytotoxic lymphocytes, the method comprising: (a) providing a cell collective comprising cytotoxic lymphocytes isolated from a biological sample obtained from a donor; (b) contacting said cell collective isolated in step (a) with a micropatterned surface as defined herein and above, to effect the activation of the cytotoxic lymphocytes in the cell collective; (c) recovering the cell collective obtained in step (b) comprising activated cytotoxic lymphocytes from the micropatterned surface, and (d) culturing the collective, in the presence of a cytokine, thereby obtaining an expanded population of cytotoxic lymphocytes in the cells collective. The method may further comprise isolating cytotoxic lymphocytes from the cells collective. Alternatively, the cytotoxic lymphocytes may be isolated by culturing and subsequent enrichment of the collective. Further, in particular some embodiments of the present disclosure provides a method for expanding/proliferating cytotoxic lymphocytes, said method comprising: (a) isolating cytotoxic lymphocytes from a biological sample obtained from a donor; (b) contacting said cytotoxic lymphocytes isolated in step (a) with a micro-pillar array functionalized with at least one ligand, i.e., to obtain an activated population of cytotoxic lymphocytes, said micro-pillar array comprising a plurality of micro-pillars essentially perpendicularly disposed on a support, each micro-pillar being characterized by a bending stiffness of between about 7 μPa*m and 450 mPa*m; and wherein said method further comprises (c) recovering the cytotoxic lymphocytes obtained in step (b), namely, the activated population of cytotoxic lymphocytes, from said at least one micro-pillar array and culturing thereof in the presence of at least one cytokine, thereby obtaining an expanded/proliferated population of cytotoxic lymphocytes.

Therefore and as detailed above, the cytotoxic lymphocytes of a collective comprising same may be contacted with the activation micropatterned surface, suitably biofunctionalized to effect said activation. The cells may then be left for a period of activation time interval, whereafter the cells are recovered and further cultures under suitable conditions, e.g., in presence of at least one cytokine, as known in the art. In other words, the present disclosure provides a method for expanding/proliferating cytotoxic lymphocytes (for example T cells, such as CD8⁺ T cells, in particular human CD8⁺ T cells), said method comprising first isolating cytotoxic lymphocytes or a heterogenous collective of cells comprising said cytotoxic lymphocytes, from a biological sample obtained from a donor (e.g., human subject), followed by contacting said isolated cytotoxic lymphocytes or a population comprising same with at least one micropatterned surface, e.g., an array, as described herein, to obtain an activated population of cytotoxic lymphocytes, and subsequently recovering the activated population of cytotoxic lymphocytes from the micro-pillar array and culturing the activated population in the presence of at least one cytokine, thereby obtaining an expanded/proliferated population of cytotoxic lymphocytes.

The presently disclosed methods are suitable for expanding/proliferating immune cells which may be used for immuno-therapeutic purposes, such as cytotoxic lymphocytes. By the term “cytotoxic lymphocytes” it is referred to a population of immune cells that exert their action via a variety of effectors, leading to a target cell death. For example, cytotoxic lymphocytes are T cells and/or natural killer (NK) cells, for example human cells. In specific embodiments, T cells are CD8⁺ T cells, CD4⁺ T cells, γδ-T cells or natural killer T cells. In particular embodiments, T cells are CD8⁺ T cells. In further particular embodiments, the T cells are human cells. Thus, the methods as disclosed herein may be carried out wherein the cytotoxic lymphocytes are CD8⁺ T cells, CD4⁺ T cells, γδ-T cells, or natural killer T cells.

As detailed above, the cells are activated by contacting the micro-pillar arrays functionalized by ligands and then proliferated by culturing under suitable conditions. By the term “activation” or “activating” in the context of the present invention it is meant triggering certain cellular events through antigen-specific receptors on the cell surface of lymphocytes, causing the cells to proliferate and differentiate into specialized effector lymphocytes. Furthermore, by the terms “expanding”, “proliferating” or “proliferation” as used herein (the terms expanding and proliferating are used herein interchangeably), it is meant increasing the numbers of, enriching a population within a certain collective, or otherwise multiplication of a specific or general cytotoxic lymphocytes' population, thereby increasing the cell numbers as compared to the initial number of cells.

As discussed briefly above, the methods of the present disclosure further comprise additional steps, for example recovering the activated population of cytotoxic lymphocytes, and/or culturing of the activated population of cytotoxic lymphocytes in the presence of a suitable growth media and supplements well known in the art (e.g., with suitable effector cytokines). Thus, as mentioned above, the method may further comprise culturing the activated population of the cytotoxic lymphocytes, which may usually be carried out in the presence of at least one cytokine, to affect the cells' expansion. Additionally, culturing of the activated cells may also be performed in presence of at least one cytokine, which may be the same or different from the cytokine used during the activation. By the term “cytokine” it is referred to a substance, such as interferon, interleukin, and growth factors, which are secreted by certain cells of the immune system and have an effect on other cells. In some embodiments the applicable cytokine is at least one of IL-2, IL-1, IL-4, IL-6, IL-7, IL-10, IL-12, IL-15, IL-18, and TNF, for example IL-2 or IL-15. Thus, the method may further comprise the culturing step as defined above in presence of at least one cytokine is selected from the group consisting of IL-1, IL-2, IL-4, IL-6, IL-7, IL-10, IL-12, IL-15, IL-18, and TNF. As mentioned above, the contacting step may also be performed in presence of at least one of these cytokines, which may be same of different from the one used in culturing. Recovering of the lymphocytes or of a collective comprising same may usually be achieved by a gentle washing of the activating micropatterned surfaces, optionally in presence of a suitable detachment assisting agent, such as an EDTA salt, and/or a mild detergent. Recovered cells may be washed with a suitable medium, e.g., the growth medium, before seeding for the culturing step.

Additionally, the culturing of the cytotoxic lymphocytes after their activation may be carried out for a pre-determined time interval. The time interval is usually dictated by the efficiency of activation and the nature of activating medium, e.g., the beads, flat surfaces, or micropatterned surfaces. It has now been found that the time frame within which the cytotoxic lymphocytes activated on micro-pillars of the present disclosure can be cultured and expanded, without development of the exhaustion signs, is significantly longer than that obtainable with the standard in the field, which is the magnetic beads such as Dinabeads™. It has now been surprisingly found that cells conventionally activated on Dinabeads™ for 24 hours climax in numbers at day 4, before rapidly declining and quiescing, whereas the cells activated on micro-pillars of the present disclosure continued proliferation until day 7. Therefore, particularly when a large number of cytotoxic lymphocytes is sought in vitro, the method may comprise culturing the activated cytotoxic lymphocytes for between 3 and 12 days, preferably between 4 and 7 days. However, if the T cells are intended for transfusion to a donor, the culturing period of the activated lymphocytes may be shortened, as the cells will continue expansion in vivo after the transfusion. It is understood that the specific time interval may be dependent on the activation duration and conditions, yet, to utilize the unexpected benefits of the presently disclosed micro-pillars, the culturing step may be longer than required for the same cells activated on Dinabeads™ at identical conditions, to start their decline, but shorter than the time required for the population itself to start its decline. The proliferation rate of the cells may be measured daily, as well known in the art. Thus, the method may comprise culturing the activated cells collective or the cytotoxic lymphocytes, for a time period of between about 3 days to about 12 days, preferably between about 4 days and about 7 days.

In specific embodiments the method for expanding/proliferating cytotoxic lymphocytes according to the present disclosure comprises isolating T cells (e.g., CD8⁺ T cells, for example human cells) from a biological sample obtained from a donor and incubating thereof on the functionalized micro-brushes for a time period of between 15 minutes and 72 hours (e.g., between 2 hours and 48 hours, for example 24 hours), optionally in presence of at least one cytokine, which may be same or different from the cytokine used in the course of the culturing step. As has been unexpectedly found by the inventors, as little as 15 minutes may be sufficient to activate the cytotoxic lymphocytes, whereas the currently known methodology uses up to 72 hours to effect the activation. In particular embodiments, the activating step may be carried out for a time interval of between 2 and 8 hours, e.g., between 3 and 6 hours.

In further specific exemplary embodiments, the activated population of cells is then detached from the micropatterned surface by pipetting, thereby allowing the cells to be collected and recovered, and optionally further rinsing of the surface, and re-cultured in complete culture medium in the presence of at least one cytokine, for example IL-2 (e.g., RPMI media supplemented with 200 units IL-2). The culturing time period may be between about 3 days to about 12 days (e.g., between about 4 days and about 7 day, or for 10 days), with suitable medium replacement frequency, e.g., replacing the medium every 3 days, with a periodic supplement of IL-2 every 7 days (200 units). Cell proliferation rate may be measured daily as well known in the art, e.g., by collecting a sample of cell cultures (e.g., 10 μl), staining the cultures with DAPI and counting the cells using a flow cytometer, or by counting the cells under microscope using hemocytometer.

Thus, in specific embodiments, provided the wherein the micropatterned surface comprises a ligand that is characterized by binding to and activating and/or stimulating at least one of the T cell receptor (TCR)-CD3 complex and/or the CD28 molecule on the surface of the cytotoxic lymphocytes. Preferably, the ligand is an anti-CD3 antibody and/or an anti-CD28 antibody. In further specific embodiments the present disclosure provides a method for expanding/proliferating cytotoxic lymphocytes comprising: (a) isolating cytotoxic lymphocytes (for example, CD8⁺ cells, such as human CD8⁺ cells) or a cell collective comprising same from a biological sample (for example, blood) obtained from a donor; (b) contacting said cytotoxic lymphocytes isolated in step (a) with at least one micro-pillar array functionalized with a first ligand characterized by binding to and activating the TCR-CD3 complex (e.g., an antibody directed to the TCR-CD3 complex) and a second ligand characterized by binding to and activating the CD28 molecule (e.g., an antibody directed to the CD28 molecule) on the surface of said cytotoxic lymphocytes, said micro-pillar array comprising a plurality of micro-pillars essentially perpendicularly disposed on a support, each micro-pillar being characterized by a length of about 1.5 to about 5 micrometers and a diameter of about 0.25 μm to about 3 μm, and a bending stiffness of between about 7 μPa*m and 450 mPa*m, and wherein said contacting is performed for a time period of 24 hours; and wherein said method further comprises: (c) recovering the cytotoxic lymphocytes obtained in step (b) from said at least one micro-pillar array and culturing thereof in the presence of IL-2, thereby obtaining an expanded/proliferated population of cytotoxic lymphocytes (e.g., expanded/proliferated population of CD8+ T cells), wherein said culturing of said cytotoxic lymphocytes is carried out for a time period of between about 4 days and about 7 days.

Cytotoxic lymphocytes may be isolated from a biological sample, where the term “biological sample” is used herein in the broadest sense and refers to a specimen or culture obtained from animals (including humans) and encompasses whole blood, peripheral blood, cord blood, bone marrow, fractionized blood containing lymphocytes, a tissue (e.g., a biopsy), or an organ. Specifically, lymphocytes may be isolated from a biological sample by obtaining blood from a human donor, by using methods well known in the art. In particular, lymphocytes may be obtained from the peripheral blood mononuclear cells (PBMCs) blood fraction. A typical process for carrying out lymphocyte separation from whole blood or fractionated blood is density gradient centrifugation, using a density gradient media (e.g. as employed in the course of the present disclosure). Centrifugation is the commonly used method for processing blood so that the cells and particles of the same size, shape and density concentrate as separate zones without convection.

As detailed above, activation and proliferation/expansion using the micro-pillar array surfaces of the present invention facilitates obtaining specific cell populations which may be transfused back to the donor of the original biological sample from which the initial cells were isolated. Therefore, the donor of the biological sample may be the patient in need of or subject to be treated by proliferated cell populations of the present invention.

The contacting step (namely, bringing into interaction the cells and the micro-pillar array, in suitable medium, e.g., by seeding the cells onto the micro-pillar array surface) may optionally be in the presence of at least one cytokine which may be the same or different from the cytokine used in the course of the proliferation (culturing) step. The cells are recovered (i.e., collected) from the micro-pillar array by any method known in the art, for example by collecting the cells using a pipette and rinsing the micro-pillar array with a suitable buffer to remove further cells therefrom after the contacting step, optionally in the present of additional agent(s).

The methods according to the present disclosure may optionally further comprise at least one additional step of modifying, and/or enriching and/or differentiating said cytotoxic lymphocytes. Thus, further provided herein methods further comprising genetically modifying said cytotoxic lymphocytes to express a chimeric antigen receptor, and/or further comprising enriching said cytotoxic lymphocytes for an antigen-specific cell population. In some specific embodiments, enriching for T cells may be performed after isolating cytotoxic lymphocytes from the biological sample, is performed, but prior to contacting with the micropatterned surface. Additionally, as described in the appended examples, genetically modifying of the cells may be performed after contacting with the micropatterned surface.

As detailed above, one of the immunotherapy approaches that may benefit from the methods disclosed herein is based on tumor-infiltrating lymphocytes (TILs), which are isolated from the tumor stroma, assayed for neoantigen-specific recognition, and expanded before being re-introduction into the patient. By the term “neoantigen” or “neo-antigen” it is referred to antigenic substances produced in tumor cells. In other words, TIL therapy is based on obtaining T cells from tumor biopsies, which are already specific against tumor antigens (which are potential targets for immune attack) and boost them with cytokines (such as IL-2) before re-introducing them into the patient's peripheral vasculature. Therefore, among others, by the methods of the present disclosure, specific cytotoxic lymphocytes populations directed to a specific neo-antigen or tumor associated antigen (TAA) may be expanded or proliferated, where the original lymphocytes are derived from TILs obtained from the subject.

In such case the methods according to the present disclosure may further comprise at least one additional step of enriching (e.g., by selection) the population of cytotoxic lymphocytes obtained from the donor with cells directed to a specific neo-antigen. In other words, the methods of the present invention may further comprise enriching the cytotoxic lymphocytes (obtained from the donor) for an antigen-specific cell population, before or after the contacting step with the micro-pillar array of the invention.

Therefore, in specific embodiments provided herein the method wherein the cytotoxic lymphocytes are tumor infiltrating lymphocytes isolated from a tumor biopsy obtained from a cancer patient in need of treatment. Thus, in specific embodiments, the present disclosure provides a method of proliferating tumor infiltrating lymphocytes (TILs), said method comprising:
(a) isolating TILs from a biological sample obtained from a donor;
(b) enriching said TILs isolated in step (a) for an antigen-specific cell population;
(c) contacting said antigen-specific cell population isolated in step (b) with at least one micro-pillar array functionalized with a first ligand characterized by binding to and activating the TCR-CD3 complex (e.g., an antibody) and a second ligand characterized by binding to and activating the CD28 molecule (e.g., an antibody) on the surface of said cytotoxic lymphocytes, said micro-pillar array comprising a plurality of micro-pillars essentially perpendicularly disposed on a support, each micro-pillar being characterized by a length of about 1.5 to about 5 micrometers and a diameter of about 0.25 μm to about 3 μm, and a bending stiffness of between about 7 μPa*m and 450 mPa*m, and wherein said contacting is performed for a time period of 24 hours; and
(d) recovering the cytotoxic lymphocytes obtained in step (b) from said at least one micro-pillar array and culturing thereof in the presence of IL-2, thereby obtaining a proliferated antigen-specific cell population of TILs, wherein said culturing of said cytotoxic lymphocytes is carried out for a time period of between about 4 days and about 7 days.

In addition, chimeric antigen receptor (CAR) T cell therapy is a type of treatment in which a patient's T cells are modified in such a manner that they will attack cancer cells. T cells are obtained from a patient's blood, then the gene for a modified “chimeric” T cell receptor that binds to a certain protein on the patient's cancer cells is added to the T cells by methods known to a skilled artisan. Large numbers of the modified CAR T cells are then grown (cultured) and administered to the patient (also the donor) by infusion.

Furthermore, by the methods of the present disclosure cytotoxic lymphocytes isolated from a donor may be modified to express chimeric antigen receptors (CARs) and further activated and proliferated before being transfused back to the donor by the methods described herein. Alternatively, expanded cytotoxic lymphocytes may be modified with CARs, prior to transfusion. In other words, the present invention provides a solution to the production of immunotherapeutic CAR T cells. This solution may further contribute to T cells production associated with already approved immunotherapies and will pave the way for the development and regulatory approval of new immunotherapies in particular for the treatment of cancer. CAR T cells may be obtained by the means as known in the art, e.g., utilizing CRISPR-Cas9 enzymatic machinery. The components may be delivered by transduction of a suitable Cas9 protein loaded with the CAR-encoding lead sequence. Alternatively, a suitable DNA fragment, particularly double-stranded DNA encoding for CRISPR-Cas9 enzymatics with CAR lead sequence, may be used. These materials may be transduced into the target cells, e.g., cytotoxic lymphocytes, either isolated or as part of cells collective isolated from a donor, e.g., by electroporating technique as known in the art. Additionally, the transfection may be performed using a suitable viral carrier, e.g., retrovirus, and specifically, a lentivirus.

In some embodiments the methods of the present disclosure further comprise genetically modifying the cytotoxic lymphocytes to express a chimeric antigen receptor (CAR), before or after the contacting step with the micro-pillar array of the invention.

Therefore, in specific embodiments provided herein the method wherein the micropatterned surface is functionalized with a first ligand characterized by binding to and activating the TCR-CD3 complex and a second ligand characterized by binding to and activating the CD28 molecule on the surface of the cytotoxic lymphocyte, the contacting being performed for a time interval of between 20 and 28 hours, wherein the cytokine of culturing step (d) is IL-2, and wherein the culturing is for a time interval of between 4 days and 7 days. Particularly, the method may be further comprising genetically modifying said cells collective comprising cytotoxic lymphocytes isolated in step (a) to express a chimeric antigen receptor (CAR). Specifically, the present disclosure further provides a method of proliferating modified cytotoxic lymphocytes, said method comprising:
(a) isolating cytotoxic lymphocytes from a biological sample obtained from a donor;
(b) genetically modifying said cytotoxic lymphocytes isolated in step (a) to express a chimeric antigen receptor (CAR);
(c) contacting said genetically modified lymphocytes obtained in step (b) with at least one micro-pillar array functionalized with a first ligand characterized by binding to and activating the TCR-CD3 complex (e.g., the first ligand is an antibody) and a second ligand characterized by binding to and activating the CD28 molecule (e.g., the second ligand is an antibody) on the surface of said cytotoxic lymphocytes, said micro-pillar array comprising a plurality of micro-pillars essentially perpendicularly disposed on a support, each micro-pillar being characterized by a length of about 1.5 to about 5 micrometers and a diameter of about 0.25 μm to about 3 μm, and a bending stiffness of between about 7 μPa*m and 450 mPa*m, and wherein said contacting is performed for a time period of 24 hours; and
(d) recovering the cytotoxic lymphocytes obtained in step (b) from said at least one micro-pillar array and culturing thereof in the presence of IL-2, thereby obtaining an obtaining a modified proliferated population of cytotoxic lymphocytes, wherein said culturing of said cytotoxic lymphocytes is carried out for a time period of between about 4 days and about 7 days.

Particularly, in further specific embodiments the method further comprises genetically modifying of the cells, e.g., comprises any one of steps of electroporating said cells in presence of a Cas9 protein bearing CAR genetic material or in presence of double-stranded DNA encoding for CRISPR-Cas9 enzymatic system bearing the CAR genetic material, or transfecting the cells with a suitable viral carrier loaded with genetic material encoding said CRISPR-Cas9 and said CAR.

By another one of its aspects the present disclosure provides a method of treating or preventing a disease in a subject in need thereof comprising administering to said subject a proliferated population of cytotoxic lymphocytes obtained by the method defined herein, wherein said cytotoxic lymphocytes are derived from said subject (e.g., T cells obtained from a human subject). The disease may be any disease the treatment thereof would benefit from transfusing or administrating to the subject a proliferated population of cytotoxic lymphocytes derived therefrom, for example cancer, or an immune-related disease (e.g., an inflammatory disease or infection).

The terms “treatment”, “treating”, or “treat”, as used herein in reference to a subject or its condition, unless the context dictates otherwise, mean at least ameliorating one or more clinical indicia (i.e., symptoms) of disease activity in a patient/subject having a pathologic disorder, e.g., cancer. Subjects in need thereof are mammalian subjects suffering from any pathologic disorder, in particular humans. To provide a “preventive treatment” is acting in a protective manner, to defend against or prevent something, especially a condition or disease, e.g., cancer. The term “preventing” as used herein in connection to a disease or a condition, also relate to reducing the incidence or prevalence of the disease or of the condition, or to reducing the severity of the condition to sub-clinical form, e.g., in relation to the cancer, to a steady disease without progression.

The effective amount, meaning the amount necessary to achieve a selected result, as well as the route of administration may be determined by a skilled person based on considerations well known in the art. In particular the proliferated population of cytotoxic lymphocytes are administered by transfusion into the subject in need thereof, namely by transferring the donated blood products into the circulatory system of the subject.

Thus, provided herein a method of treating or preventing a disease in a subject in need thereof, the method comprising administering to the subject an effective amount cytotoxic lymphocytes obtained by using a micropatterned surface as described herein, or obtainable by the method for activating and/or expanding a cell population comprising cytotoxic lymphocytes, wherein the cytotoxic lymphocytes are derived from the same subject, and wherein the disease is a cancer, an inflammatory disease, or an infection.

In specific embodiment the proliferated population of cytotoxic lymphocytes are CAR T cells. In various other embodiment the proliferated population of cytotoxic lymphocytes are TILs. In particular embodiments, the expanded population of cytotoxic lymphocytes has been expanded for a time interval of between 4 and 7 days.

By a further embodiment the present disclosure provides a population of cytotoxic lymphocytes (e.g., T cells, such as human T cells) obtained by the methods herein described for use in treatment or prevention of a disease in said donor (i.e., disease in the subject from which the lymphocytes were obtained), for example in treatment of cancer.

Advantageously, the micropatterned surfaces as generally described herein, may be present or integrally formed with a medical device, e.g., the medical devices for use in manipulating cells and blood constituents. For example, the micropatterned surfaces may be present in or as part of a bioreactor that may be used to activate the cells, e.g., PBMC. The cells, particularly the blood cells, may be derived from a donor, or from a subject in need of a treatment mediated by activated and/or expanded lymphocyte population, as elaborated below. The medical device comprising the micropatterned surfaces, particularly the pre-biofunctionalized micropatterned surfaces, may also be the tubing and the plastic containers used for the transfusion of blood products, particularly, blood sachets. In some preferable embodiments, the device is an insert fitting a tissue culture plate or tissue culture bottle.

Thus, in a further aspect provided herein a medical device comprising a micropatterned surface as generally described herein.

By a further aspect thereof the present disclosure provides a micro-pillar array functionalized with at least one ligand, for use in proliferating and/or enriching a population of cytotoxic lymphocytes (e.g., T cells, such as human T cells), wherein the micro-pillar array is essentially as described herein. In preferred embodiments, the micro-array comprises a plurality of pillars essentially perpendicularly disposed on a support, each micro-pillar being characterized by a bending stiffness of between about 7 μPa*m and 450 mPa*m. In specific embodiments the micro-pillars are characterized by a length of about of between 1.5 and about 5 micrometers and a diameter of about 0.25 μm to about 3 μm. Preferably, the micro-pillars are contained in a density of between about 50,000 to about 275,000 micro-pillars per square millimeter. The micro-pillars preferably comprise PDMS, and are coated with a ligand that is characterized by binding to and activating and/or stimulating at least one of the TCR-CD3 complex and/or the CD28 molecule on the surface of a cytotoxic lymphocyte, and the ligands are coated onto said micro-pillars with a linking moiety comprising a siloxy residue.

Still further aspect of the present disclosure provides a kit for practicing the methods according to the present disclosure the kit comprising a micro-pillar array and instructions for using the same. In some embodiments the kit as herein defined is wherein said micro-pillar array consists essentially of cross-linked polydimethyl siloxane, and wherein said instructions contain directions to any one of the following steps: i) exposing said micro-pillar array to oxygen plasma; ii) providing a solution of (3-aminopropyl)-triethoxysilane to said micro-pillar array, and/or iii) providing a solution of at least one ligand to said micro-pillar array.

In other embodiments the kit according to the present disclosure further comprises at least one of a) a solution of or a weighed quantity for reconstitution of (3-aminopropyl)-triethoxysilane, preferably in a solvent comprising ethanol, and/or b) a solution of or a weighed quantity for reconstitution of anti-CD3 antibody, preferably in a biologically acceptable medium, and/or c) a solution of or a weighed quantity for reconstitution of anti-CD28 antibody, preferably in a biologically acceptable medium. In various embodiments the kit further comprises a solution of or a weighed quantity for reconstitution of IL-2, preferably in a biologically acceptable medium. In further embodiments all components of the kit as herein defined, optionally excluding said instructions, are sterile.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to +10%.

The terms “comprises”, “comprising”, “includes”, “including”, “having” and their conjugates mean “including but not limited to”. This term encompasses the terms “consisting of” and “consisting essentially of”.

It is appreciated that certain features of the invention, which are, for brevity, described in the context of separate embodiments, may also be provided in combination in a single embodiment. Conversely, combinations of various features of the invention, which are, for clarity and demonstration, described in the context of a single embodiment, may also be provided separately or in any suitable sub-combination, or as suitable in any other described embodiment of the invention. Additionally, various features according to the invention as described herein for one aspect are applicable mutatis mutandis for the corresponding features in other aspects, according to the teachings herein. Preferred embodiments described according to one aspect should be regarded as similarly preferred embodiments in other aspects, unless the context explicitly prescribes otherwise.

It must be noted that, as used in this specification and the appended claims, the singular forms “a”, “an” and “the” include plural referents unless the content clearly dictates otherwise.

As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

Various embodiments and aspects of the present invention as delineated hereinabove and as claimed in the claims section below find experimental support in the following examples. The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Reference is now made to the following examples, which together with the above descriptions illustrate some embodiments of the invention in a non-limiting fashion.

### Experimental Procedures

### Microscopy

Confocal and z-stack images of cells on the various surfaces were acquired using a Zeiss LSM 880 confocal microscope and quantified using the Fiji imaging software. SEM images were acquired using a FEI Verios XHR 460L Scanning Electron Microscope. The surfaces with cells were fixed, dried in Critical Point Drier (CPD), sputter coated with 5 nm Au, and the SEM imaging was done at a voltage of 3 kv and current of 0.10 nA, and pseudo colored using Adobe™ Illustrator™.

### Statistics

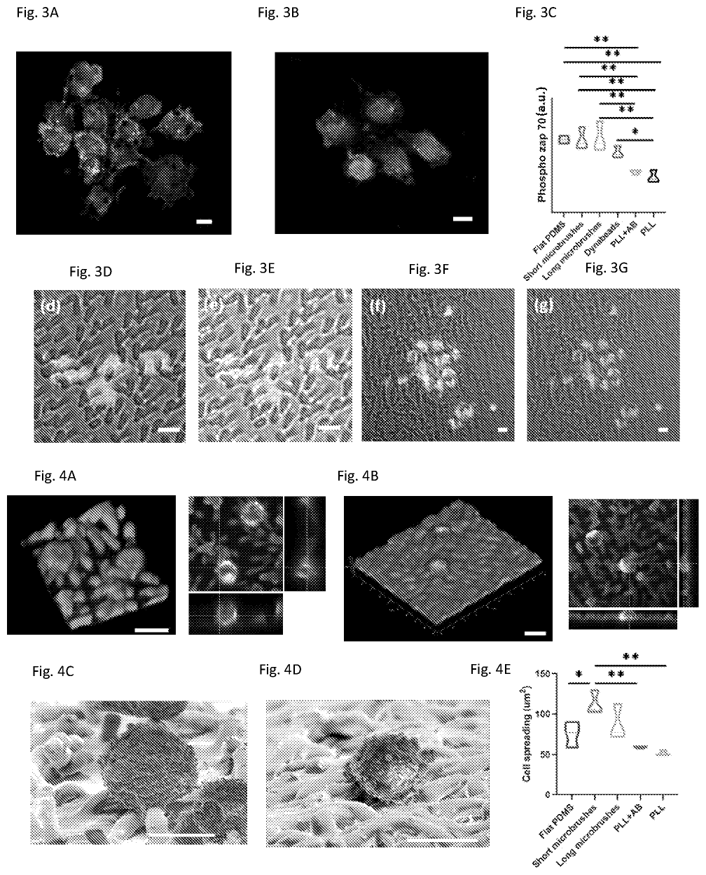

All biological experiments were performed in triplicates. For image data, 30 to 60 regions were imaged and averaged for quantification. In FIGS. 2-5, means with SD are shown. Unless specified otherwise, statistical analyses were performed by analysis of variance ANOVA with Tukey's multiple comparison post hoc test. Some of the data was z-score-normalized, as specified. The results were considered to be significantly different for p<0.05.

### Flow Cytometry

For flow cytometry measurements, 50,000 cells were used per well. The cells were washed with ×1PAF solution, composed of 1% Phosphate buffered saline (PBS), 0.05% sodium azide and 2% fetal bovine serum (FBS), and seeded in 96 well plate. The cells were then stained with respective fluorophore-conjugated antibodies as specified in the relevant sections, in 1:100 dilution (1 μg/mL), and incubated for 30 minutes on ice. Thereafter, the cells were washed, and the dead cells were stained with DAPI (1 μg/ml). All the samples were analyzed in Beckman CytoFLEX L flow cytometercytometer (Violet-blue-Yellow Green-Red-IR configuration). For analysis, fraction of CD3-positive cells was calculated, and CD3-positive cells were then analyzed for staining with the other antibodies employed for staining, as elaborated in specific examples.

### Example 1

### T Cells Adherence to and Activation by Functionalized Micro-Brushes

In order to evaluate the stimulatory effect on human T cells by elastic polydimethylsiloxane (PDMS) “micro-brushes” or pillars, primary human T cells were stimulated on micro-brushes of pillars, as detailed herein below.

PDMS micro-brushes were fabricated via the double replication method, as elaborated below, from a positive silicon mold, with two different pillar lengths, of about 5 μm and 10 μm, which are also termed hereafter “short” and “long” micro-brushes, respectively.

Silicon master molds, containing micro-brushes having periodicity of 4 microns (μm), diameter of 1 micron, and length of either 5 microns (also referred to herein as “short micro-brushes”) or 10 microns (also referred to herein as “long micro-brushes”) were created using electron beam lithography and Bosch process, as described elsewhere (B. Pokroy, et al. Science 2009, 323, 237). The silicon master molds were first coated with trichloro (1H,1H,2H,2H-perfluorooctyl) silane (Sigma-Aldrich) overnight in vacuum to obtain an anti-adhesive hydrophobic layer on the silicon micro pillars. Then, Sylgard 184 PDMS kit (DOW) was mixed (containing 10:1 PDMS: hardener) and poured onto the surface of the silicon micro pillars, degassed and then cured for an hour at 60° C. The cured PDMS was then peeled off from the silicon and coated with anti-adhesive coating of trichloro (1H,1H,2H,2H-perfluorooctyl) silane (overnight, in vacuum). This negative PDMS mold containing holes, was further used for double replication by pouring PDMS (10:1 PDMS: hardener) and curing at 60° C., overnight. The cured PDMS was then peeled off from the negative mold to obtain PDMS micro-brushes. These micro-brushes were coded “1-4-5[1:10]” for short and “1-4-10[1:10]” for long, the code designating “diameter×pitch (density)×length [hardener ratio]”.

The pillars, as visualized for example in the SEM micrograph FIG. 1B, have a diameter of about 1 μm (in both the “short” and “long” micro-brushes), and were arranged in rectangular arrays with a periodicity of 4 μm. The resulting elastomer, had an elastic modulus of ˜2 MPa.

In order to provide the activating and costimulatory biochemical signals necessary for T cell activation, the surface of micro-brushes was functionalized with α-CD3 and α-CD28 antibodies (i.e., antibodies directed to CD3 and CD28, respectively), as follows.

The PDMS surfaces (micro-brushes) were functionalized with a mixture of the activating ligands α-CD3 and α-CD28 (i.e., antibodies directed to CD3 and CD28, respectively). The samples (micro-brushes) were first treated with oxygen plasma for 5 seconds (Harrick PDC32G) and then modified with (3-aminopropyl)-triethoxysilane (APTES, Sigma-Aldrich) by immersing into a 10% ethanolic APTES solution for 10 minutes, rinsing with ethanol, and baking for 10 min in an oven at 60° C., as previously described (P. V'kovski, et al., 2019, Elife 8: e42037). The samples were then incubated overnight at 4° C. in a 2 μg/ml solution of a mixture of the antibodies α-CD3 and α-CD28 (1 μg/ml individual concentration each) in sterile phosphate buffered saline (PBS), then rinsed three times and stored in sterile PBS until use.

In the experiments conducted as described in the present example, the following types of control stimulatory surfaces were used: (i) Flat PDMS functionalized with α-CD3 and α-CD28 antibodies, based a material having the same elasticity as the micro-brushes, but devoid of any stimulating micro-topology (termed “flat PDMS” in the Figures); (ii) rigid glass slides (i.e., microscope glass slides 26×76 mm, with thickness of 1.0-1.2 mm) functionalized with activating and costimulatory antibodies via a pro-adhesive poly-l-lysine (PLL) layer (termed “PLL+AB” in the Figures); (iii) rigid glass slides coated with PLL only, lacking any activating stimuli (termed “PLL” in the Figures); and commercial magnetic beads (Dynabeads™) functionalized with α-CD3 and α-CD28 (termed “Dynabeads” in the Figures). The control surfaces were prepared as follows.

Flat PDMS functionalized with α-CD3 and α-CD28 antibodies was prepared using a flat plastic petri dish functionalized as described for the micro-brushes. Rigid glass slides (microscope glass slides 26×76 mm, thickness 1.0-1.2 mm) were functionalized with activating and costimulatory antibodies via a pro-adhesive poly-l-lysine (PLL) layer and rigid glass slides coated with PLL only were prepared as previously described (E. Toledo et al. Science Adv. 7, eabc1640 2021). Commercial magnetic beads (Dynabeads™) were functionalized with α-CD3 and α-CD28 as previously described (E. Toledo et al, Science Advances, 2021, 7, abc1640).

First, it was studied whether the micro-brush topology affects the cytotoxicity of CD8⁺ T cells. To this end, primary human CD8⁺ T cells were isolated from the peripheral blood of a healthy donor, as follows. Following approval by the Institutional Review Board of Ben-Gurion University of the Negev, CD8⁺ T cells were isolated from fresh blood of a healthy adult volunteer donor, recruited by written informed consent. Peripheral blood mononuclear cells (PBMCs) were isolated from the blood using the FICOL gradient. First, blood was diluted in 2% fetal bovine serum (FBS) at a 1:1 ratio, then loaded on FICOL gradient and centrifuged at 1200 g-force with no breaks or acceleration. The collected PBMCs were cultured in complete RPMI media with 10% human serum supplemented with 200 units of the cytokine IL-2 and 50 ng of an anti-CD3 antibody for 2 days. Finally, CD8⁺ T cells were isolated with a positive selection of CD8⁺ beads using a magnetic column according to the company instructions (Life technologies). CD8⁺ T cells were continuously cultured in complete RPMI media with 10% human serum supplemented with 200 units of IL-2.

Cultured CD8⁺ T cells were seeded onto functionalized PDMS surfaces in growth medium containing <2% serum and 50 units of IL-2 and left to adhere for 3 hours. After incubation, samples were rinsed twice in PBS to remove the non-adherent cells and the adherent cells were then fixed with 4% PFA. Adherence of a T cell to the resulting functionalized micro-brush is shown in FIG. 1A (schematically) and in FIG. 1B (a SEM micrograph). For SEM imaging, after the fixation in 4% PFA, cells were dehydrated with 4 successive ethanol baths, dried using critical point drying and plated with 1-2 nm of gold to limit surface charging during imaging.

The T-cells were stimulated on the micro-brushes and the control surfaces for three (3) hours were also tested for activation. Thus, the amount of exposed lysosome-associated membrane protein-1 (LAMP-1, also known as CD107a) was assessed in the tested cells. In activated T cells, lytic granules containing granzymes and perforins, designed to induce death in target cells, diffuse towards the immune synapse, where they merge with the membrane and release their lytic content into the synaptic space. Amongst the structural proteins found in lytic granules is CD107a, which is brought to the surface of the membrane during the degranulation and is thus commonly used as a quantitative marker for the activation of T cells. The amount of expressed CD107a in each of the tested T cell populations was therefore assessed by immunostaining by fluorescently labeled anti-CD107a antibody and by measuring its average fluorescence intensity per cell, as follows.

Cultured CD8⁺ T cells were seeded onto PDMS and control surfaces in growth medium containing <2% serum and 50 units of IL-2 and left to adhere for 3 hours. Then, samples were rinsed twice in PBS to remove the non-adherent cells and the adherent cells were fixed with 4% paraformaldehyde (PFA) and blocked with 5% bovine serum albumin (BSA, Tocris bioscience) in PBS. The actin cytoskeleton was stained with Alexa Fluor® 555 phalloidin (Invitrogen), and the nuclei were stained by mounting the samples with ProLong® Gold antifade reagent containing 4,6-diamidino-2-phenylindole (DAPI) (both from Life Technologies). For the imaging of CD107a (a degranulation marker), the cultured T cells were seeded as mentioned above. The medium was supplemented with Allophycocyanin anti-human CD107a (1:1000 v/v in IL-2 poor medium, Biolegend) and was left to adhere for 3 hours. The samples were rinsed twice in PBS, fixed with 4% PFA, and then directly stained with Alexa Fluor® 555 phalloidin without permeabilization to prevent damage to the cell membrane. Finally, the nuclei were stained by mounting the samples with ProLong® Gold antifade reagent containing DAPI.

FIG. 2A and FIG. 2B show top-view and z-stack confocal images of T cells stimulated on short micro-brushes. CD107a (green in color and bright spots in greyscale) is clearly seen in a significant part of the cells stimulated on the short brushes. FIG. 2C shows the average amount of degranulated CD107a signal per cell stimulated on micro-brushes and the control surfaces. As shown in FIG. 2C, cells seeded on short PDMS micro-brushes (“short PDMS pillars”) secreted CD107a at the highest level, indicating the maximal activation of T cells was achieved on the short micro-brushes, which was significantly higher than that observed in all other surfaces. In particular, the level of CD107a expression of T cells on short PDMS micro-brushes was 2.5 times higher as compared to that produced by T cells incubated with the Dynabeads™. Long PDMS micro-brushes (“long PDMS pillars”) also induced higher activation signaling as compared to Dynabeads™, as well as compared to the flat PDMS functionalized with the antibodies (“flat PDMS”). The lowest level of CD107a expression was obtained for the rigid control surfaces lacking activating and costimulatory antibodies (“PLL”). Interestingly, whereas the difference between Dynabeads™ and the functionalized short micro-brush was three folds in favor of the micro-brush, the difference in the CD107a signal between Dynabeads™ and the rigid surface lacking the antibodies (“PLL”) was minor and not statistically significant, suggesting that the combined effect of elasticity and micro-topology on the activation of T cells is at least as potent as that of the presence of activating and costimulatory molecules on a rigid activating surface.

The positive effect of the micro-brush topology on T cell activation was further confirmed by assessing the secretion of interferon-gamma (IFN-γ) which has important roles in tissue homeostasis, immune and inflammatory responses, and tumor immuno-surveillances well as cytostatic, pro-apoptotic, and anti-proliferative functions in tumor immune therapies. In order to quantify the secretion of IFN-γ, T cells were seeded on the micro-brushes and control surfaces, as well as incubated with Dynabeads™, for 24 hours, and the supernatant was analyzed by ELISA (vide infra). In order to maintain the same conditions for all the cells seeded onto different surfaces, including the controls, they were mounted in chambers that exposed a constant surface area for all the cells, as well as constant volume. These chambers were made from PDMS by curing and then carving out a well-shaped chamber that could incorporate the volume of 300 μl.

IFN-γ production after stimulation of T cells on PDMS micro-brushes and control surfaces was determined by a standard sandwich immunoassay (ELISA). Functionalized PDMS surfaces were incubated at 37° C. with T cell in growth medium containing 50 units of IL-2 for 24 hours. After the incubation period, the cells were collected by re-suspending using a pipette and centrifuged at 1500 rpm (a G force of 126) for 5 minutes at 4° C. The supernatant was then collected and analyzed for IFNγ detection. Prior to the test, 96-well plates were coated with 100 μl/well of anti-human IFNγ (capture antibody, 1 μg/well, Biolegend). Blocking (PBS+10% FCS) was applied for 2 hours at room temperature. Following 2 hours of incubation with the supernatant, biotin anti-IFNγ detection mAb (2 μg/well, Biolegend) was added to each well. For detection, streptavidin-HRP (Jackson, 016-030-084) diluted to 1:1000 was added for 30 min. Following copious washing, 10 μl per well of 3,3′,5,5′-Tetramethylbenzidine (TMB substrate, DAKO, S1599) was added to each well and left to react for 10-15 minutes. After the addition of a stop solution, the optical density in each well was read at 450 nm (Thermo Electron Corporation Multiskan Spectrum). Between each step, wells were washed five times with PBS containing 0.05% Tween 20 (PBST).

FIG. 2D shows the IFN-γ amount vs. the type of activating surface, the trend that mirrors the one observed for the expression level of CD107a. In other words, the highest IFN-γ level was produced by T cells activated on functionalized short micro-brushes. Of note, long micro-brushes caused a higher release of INF-γ than flat PDMS, Dynabeads™, and the functionalized rigid surfaces, yet the value was lower compared to that produced on the short micro-brushes. Overall, Dynabeads™ caused a lower INF-γ secretion by T cells than all of the tested elastomeric (micro-pillar) surfaces.

### Example 2

### Early TCR Signaling on Functionalized Micro-Brushes

In order to understand the mechanism underlying the enhanced activation of T cells by elastic micro-brushes and, specifically, to assess whether the brush topology per se modifies TCR function, early TCR signaling was monitored. In T cells, the engagement of TCR by its ligand is followed by the phosphorylation of its immune receptor activation motif (ITAM), to which the cytoplasmic tyrosine kinase ZAP-70 is recruited and then phosphorylated. ZAP-70 phosphorylation, in turn, initiates downstream signal propagation and is, therefore, broadly used as a marker for the early signaling event. To assess the intensity of early TCR signaling, T cells were immobilized on the micro-brushes and control surfaces for 15 minutes, fixed, and stained against ZAP-70 phosphatase by indirect immunofluorescent staining, as follows.

Cultured T cells were seeded on the various tested substrates in growth medium containing <2% serum and 50 units of IL-2 and left to adhere for 15 minutes. The cells were then fixed with 4% paraformaldehyde (PFA, Bio-lab) for 15 minutes at 4° C. Next, the samples were gently rinsed with PBS, permeabilized with 0.1% Triton ×100 in PBS for 3 minutes at 4° C., and ice-cold methanol for 10 minutes at −20° C., and then blocked with 2% BSA for 1 hour at 37° C. Phospho-Zap-70 (1:50 in 2% BSA in PBS, Cell Signaling) was added to the samples and kept overnight at 4° C. The samples were then rinsed with PBS and incubated with anti-rabbit Alexa Fluor® 647 and Alexa Fluor® 555 phalloidin (1:1000 and 1:40 respectively, both from local supplier of Invitrogen®) at room temperature for 1 hour. Finally, the samples were rinsed with PBS twice, once in deionized water and then the nuclei were stained by mounting the samples with ProLong® Gold antifade reagent containing DAPI.

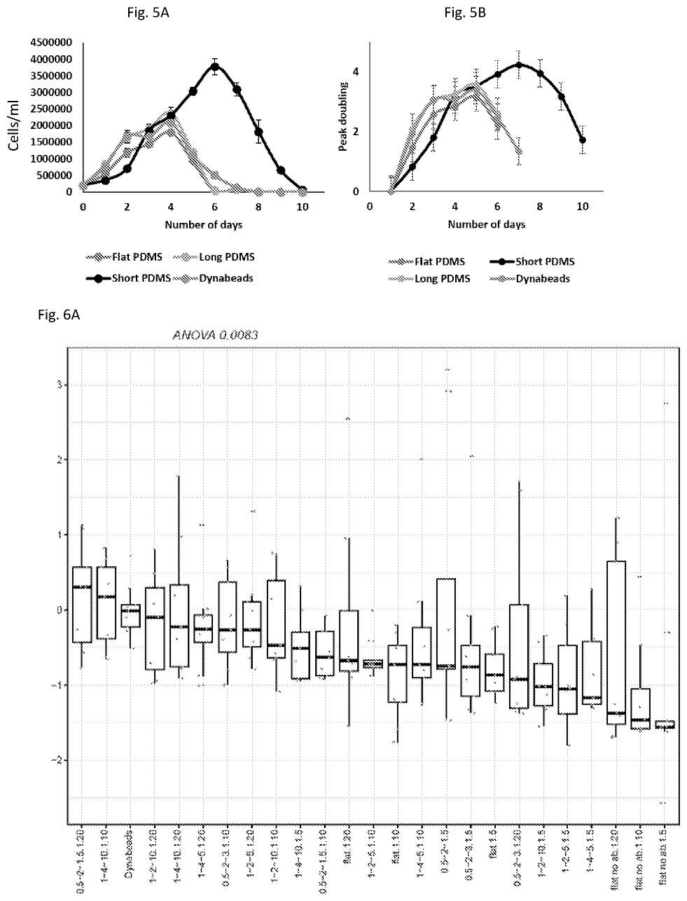

The intensity of phosphorylation of ZAP-70 was quantified using confocal microscopy. FIG. 3A and FIG. 3B show the phosphorylation of ZAP-70 in T cells on short and long (functionalized) micro-brushes, respectively, and FIG. 3C shows the average signal of phospho-ZAP-70 per cells on the brushes and the control surfaces.

As shown in FIG. 3C, the highest level of ZAP-70 was obtained for all PDMS-based surfaces (i.e., short and long micro-brushes), with no difference between them, while the lowest level was obtained for the PLL coated glass slides (“PLL”). Dynabeads™ produced early signaling that was higher than that obtained on the PLL-coated surfaces (i.e., PLL+AB and “PLL”), yet lower than that obtained on PDMS-based surfaces, although with no statistical significance. The higher early activation demonstrated for T cells incubated on PDMS-based surfaces after as little as 15 minutes of contact proposes that the brush topology per se modulates, at least partially, TCR function.

In addition, the intracellular location of ZAP-70 was also analyzed by immunofluorescence in T cells. The visualization with confocal microscopy (FIG. 3D-G) alongside with Bright-field channel showed that ZAP-70 was not localized to the interaction point of T cells and the micro-brushes, whereas it was found to be dispersed all over the T cell.

### Example 3

Mechanical Interaction of T Cells with the Micro-Brushes

In order to analyze the mechanical interaction of T cells with the micro-brushes, the membranes of the T cells were stained, and imaged using z-stack confocal microscopy, as described below.

Cultured CD8⁺ T cells were seeded onto functionalized PDMS surfaces in growth medium containing <2% serum and 50 units of IL-2 and left to adhere for 3 hours. The medium was supplemented with CellMask™ green plasma membrane stain to a final dilution of 1:1000 (Invitrogen) 10 minutes before the end of the incubation (adherence) period. After incubation, samples were rinsed twice in PBS to remove the non-adherent cells and the adherent cells were then fixed with 4% PFA. The surface bound mouse α-CD3 and α-CD28 antibodies were fluorescently labelled by incubation in goat anti-mouse Alexa 647 conjugate (Thermo Fisher) in PBS supplemented with 2% BSA overnight at 4° C. Finally, samples were mounted with ProLong® Gold antifade reagent containing DAPI to stain the nuclei. For SEM imaging, cells were incubated on the surface as previously described. After fixation in 4% PFA, cells were dehydrated with 4 successive ethanol baths, dried using critical point drying and plated with 1-2 nm of gold to limit surface charging during imaging.

Apparently, cells adhered to the short micro-brush surface (FIG. 4A) seem to be more spread as compared to the cells adhered to the long micro-brush surface (FIG. 4B), with the average values corresponding to the dashed lines. In addition, as shown in FIG. 4A, the cross section of z-stack clearly reveals that the pillars of the short micro-brushes invaginate the cell membrane, and that the cell membrane, in turn, protrudes into the interstices between the pillars. Finally, it is clearly seen that the pillars contacted by the cells are bent toward the cell center, suggesting that these pillars complied with the centripetal forces applied by the cells. Interestingly, as shown in FIG. 4B, all these features are missing from the cells' interaction with the long micro-brush surface, which seem to lie on surface and do not interact mechanically with the cells.

To confirm the above observations of the confocal microscope, the fixed cells were dried in a critical point drier, coated with a few nanometers (about 5 nm) of gold (Au), and imaged with a scanning electron microscope (SEM) as described above. Micrographs are shown in FIG. 4C and FIG. 4D for the short and long micro-brushes, respectively. These SEM micrographs reveal the three-dimensional (3D) images of the cell interaction with the two types of surfaces (which cannot be revealed by optical microscopy). First, it is clearly seen that T cells physically interact with the short micro-brushes (FIG. 4C). The pillars of the brushes produce invaginations in the cell membrane, of about 1-2 microns in depth. These invaginations are used as “contact anchors” through which the cells apply forces on the pillars, as deduced from the deflection of the pillars. Furthermore, most of the pillars contacted by the cells are bent toward the cell center, suggesting that they complied to the forces applied by the cells during the early activation stage. Contrary to the image observed in the case of short brushes, the long brushes failed to provide the anchoring contact points for T cells, as the long pillars were stuck to the surface and likely formed a “carpet” or a “mesh” formed by the reclined pillars (FIG. 4D). This was likely the reason why the shape of T cells on these surfaces was more rounded and lacked invaginations.

Finally, the interaction of T cells with the activating surface was quantified in terms of their projected areas (FIG. 4E). Here, T cells stimulated on the short micro-brushes produced the largest area. This data largely agrees with the observation from SEM and z-stack confocal microscopy that the topology of the short brushes, which promotes the protrusion of the cell membrane into the interstices between the pillars, is also a trigger for the spreading of the cells.

### Example 4

### T Cell Activation and Proliferation on Micro-Brushes

In vivo, T cell activation leads to T-cell proliferation, with the purpose of clonal selection and expansion of antigen-specific T cells. Ex vivo, T cell activation is an essential step for T cell proliferation with the purpose of obtaining T cell quantities that are sufficient for use, for example, in adoptive immunotherapy. In order to verify that the micro-brush-induced T cell activation can also produce the stimulation required for T cell proliferation (interchangeably used herein with term expansion), proliferation assays were carried out. In these assays, T cells were incubated on the micro-brushes and the control surfaces for 24 hours, were collected by pipetting, and re-cultured in complete culture medium for additional 10 days, as follows.

T cells were incubated on the micro-brushes and control surfaces for 24 hours, detached and collected by pipetting, and re-cultured in complete culture medium (RPMI media 10% human serum-supplemented with 200U IL-2) for additional 10 days, replacing the medium every 3 days, with a periodic supplement of IL-2 every 7 days (200 units). Cell proliferation rate was measured daily by collecting cell cultures (10 μl), staining the cultures with DAPI and counting the cells using a flow cytometer (Beckman).

Antibody-functionalized flat PDMS and functionalized Dynabeads™ were used as control surfaces in the proliferation assays since they showed the highest activation among the control surfaces in the activation assays. Cell proliferation rate was measured daily by collecting 10 μl of cell cultures, staining the culture with methylene blue and counting the cells using a flow cytometer.

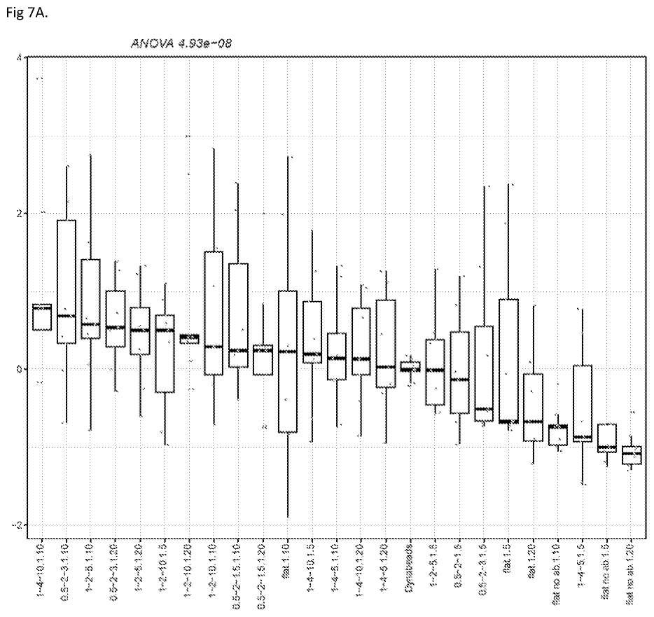

FIG. 5A shows the cell count vs. time in days for the different activating surfaces. As demonstrated in FIG. 5A, T cells incubated on short PDMS micro-brushes showed the highest proliferation rate as compared to the other surfaces, among which are Dynabeads™, the most prominent platform for T cell proliferation currently used. Remarkably, for Dynabeads™, flat PDMS and the long micro-brushes, T cell numbers peaked at day 4, and rapidly declined afterwards, suggesting the exhaustion of the T cells after day 4. On the contrary, proliferation of T cells activated on the short micro-brushes continued increasing at a rapid rate and peaked at day 6 with value exceeding 3.5×10⁶ Cells/ml. After day 6, the amount of T cells activated on the short micro-brushes gradually decreased until day 10. As shown in FIG. 5A, T cells activated on the short micro-brushes showed not only a longer proliferation time, but also proliferated to a substantially higher amount compared to those activated on other surfaces. FIG. 5B shows an analysis of population doubling vs. time for the above results. It is clearly seen that at the peak of the proliferation, short micro-brush produced a population doubling index which was 30% higher than that obtained on all other surfaces at their population peak.

Without being bound by a theory, these findings demonstrate that the micro-topology of an elastic stimulating surface is an important regulator not only of the activation of T cells but also of their proliferation. Taken together, the above findings show that elastic activating surfaces with controllably structured micro-topology have great potential on controlled, ex vivo activation of T cells for therapeutic applications.

### Example 5

Effect of the Micro-Topology of Micro-Brushes on the Activation, Proliferation, and Exhaustion of Lentivirus-Transduced CAR T Cells from Peripheral Blood Mononuclear Cells Population

To evaluate the potential of the micro-brushes to activate the T cells in diverse PBMC population and to evaluate the micro-topology effects thereof, series of activating surfaces (i.e., micro-brushes) were prepared, as generally described in Example 1 above. The activating surfaces differed by the villi (micro-pillar) thickness, pitch (density of the micro-pillars), length of the villi, and the composition of the PDMS used for the manufacturing thereof. Specifically, ratios of 1:5, 1:10, and 1:20 of the hardener to polymer, were used. These ratios, after standard curing at 60° C., yield bulk moduli of 3 MPa, 1.5 MPa, and 0.5 MPa, respectively. Two villi thickness that were tested were 0.5 μm and 1 μm, two pitch distances were 2 μm and 4 μm, and four lengths, namely, 1.5 μm, 3 μm, 5 μm, and 10 μm, in three polymer composition ratios and hence different mechanical properties, giving rise to 18 selected combinations. Additionally, coated and uncoated flat surfaces of the same materials were used, alongside the state-of-the-art product for cells' expansion, the Dynabeads™, giving additional 7 test groups.

Specifically, the following activating surfaces were prepared:

| | | |
|---|---|---|
| 0.5-2-1.5[1:5] | 1-2-5[1:5] | 1-4-5[1:5] |
| 0.5-2-1.5[1:10] | 1-2-5[1:10] | 1-4-5[1:10] |
| 0.5-2-1.5[1:20] | 1-2-5[1:20] | 1-4-5[1:20] |
| 0.5-2-3[1:5] | 1-2-10[1:5] | 1-4-10[1:5] |
| 0.5-2-3[1:10] | 1-2-10[1:10] | 1-4-10[1:10] |
| 0.5-2-3[1:20] | 1-2-10[1:20] | 1-4-10[1:20] |

Noteworthy, that surfaces 1-4-5[1:10] and Jan. 4, 2010 [1:10] were identical to the surfaces used in the examples 1˜4 above.

The PBMC were obtained from three healthy donors, recruited by written informed consent, following approval by the Institutional Review Board of Ben-Gurion University of the Negev. Peripheral blood mononuclear cells (PBMCs) were isolated from the blood using the FICOL gradient. First, blood was diluted with PBS augmented with 2% fetal bovine serum (FBS), at a 1:1 ratio, then loaded on FICOL gradient and centrifuged at 16° C. at 1200 g-force with no breaks or acceleration. The PBMC were collected as the middle disc and a small portion of the underlying phase but taking care not to withdraw the pellet, washed three times with at least 1:2 with PBS 2% FBS at room temperature and sedimented at 500 g. The cells were finally suspended in the final medium in a ratio of 2 mL per every 7 mL of collected blood, counted, and diluted with the medium to final concentration of 1×10⁶ cells per mL. The medium was NutriT™ medium (#Sartourious—Beit Haemek, Cat no.: 05-F3F2111-1K), supplemented with 200 units per mL of the cytokine IL-2 (Cat no.: 200-02-500UG, PeproTech, Cranbury, NJ USA), Pen/Strep (1%), sodium pyruvate (1 mM), NEAA (non-essential amino acids) (0.1 mM), Hepes (10 mM) and L-glutamine (2 mM).

The sterile tested surfaces were prepared for the cell experiments as follows. Obtained micro-pillar surfaces were cut before APTES functionalization to the desired size of 10 mm fitting the 48-well tissue culture plate, using a circular blade. Then, the surfaces were activated in UV ozone for 5 minutes, and APTES solution in ethanol was added for 30 minutes, washes with ethanol three times, and cured at 60 degrees for 20 minutes. The discs were kept in ethanol to ensure sterile conditions during handling. Once in the cell culture hood, the ethanol was aspirated and the discs were washed three with sterile PBS, each lasting 2-3 minutes, thus eliminating any remaining ethanol from the PDMS. After the washing, the samples were incubated with a 2 μg/mL of 1:1 mixture of anti-CD3 and anti-CD28 antibodies in sterile PBS: ca. 30-50 μL of the solution was added per sample in a sterile 6-well plate, ensuring that the amount is sufficient, and the liquid meniscus covers the entire disc surface. The 6-well plate was sealed with Parafilm™, and the incubation with the antibodies was performed overnight at 4° C. Thereafter, the surfaces were transferred to labeled 48-well plates, ensuring that the area of interest is facing upwards, and wash three times with sterile PBS to remove any excess of antibody.

The PBMC were activated on the tested surfaces in the 48-well plates, in 600 μL aliquots, equaling to about 600,000 cells per well, at 37° C., in 5% CO₂ humidified atmosphere for 24 hours.

Chimeric antigen receptor was transduced to the PBMC using lentivirus, as follows. The pre-activated PMBC were gently detached from the activating surfaces and transferred to the new sterile 48-well plate, by four-fold aspiration-return cycle of 550 μL (of 600 total) of the culturing medium, and terminal aspiration of the medium and transferring it to the new plate. The cells were sedimented using centrifuge at 500 g for 5 minutes at room temperature, 550 μL of the old medium was carefully aspirated and substituted for the fresh 100 μL of warm complete NutriT™ medium, 400 μL of lentivirus-containing the anti-HER2 3^{rd} generation CAR (supplemented with IL-2 to 200 IU/ml)+BX795 (InVivoGen, Cat no.: 6143-43-01, final concentration of 6 micromolar). The cells were further cultured in presence of the lentivirus at 37° C., in 5% CO₂ humidified atmosphere for additional 24 hours, which is up to 48 hours post extraction from the donor.

Proliferation, activation, and exhaustion of the T cells in the PMBC population was monitored as follows. On Day 3, 48 hours post extraction and 24 hours post transfection, the medium was carefully changed by centrifuging at 500 g for 5 minutes at room temperature, removing carefully 450 μL of the old medium and substituting with fresh pre-warmed complete NutriT™, and left culturing for additional 24 hours, which is up to 72 hours post extraction from the donor.

On Day 4, which is at about 73 hours post extraction from the donor, the cells were counted using CellDrop FL cell counter (LabMark, Czechia), and cells were split into multiple wells on 24-well plate with density of up to 1×10⁶ cells per well, or left proliferating in original plates if the density was below 5×10⁵ per well. The cells were followed up to Day 7, when all cells were counted.

For evaluation of the proliferation, cells were counted using trypan blue stain (1:1 dilution) on DeNovix CellDrop FL cell counter (Hylabs) machine.

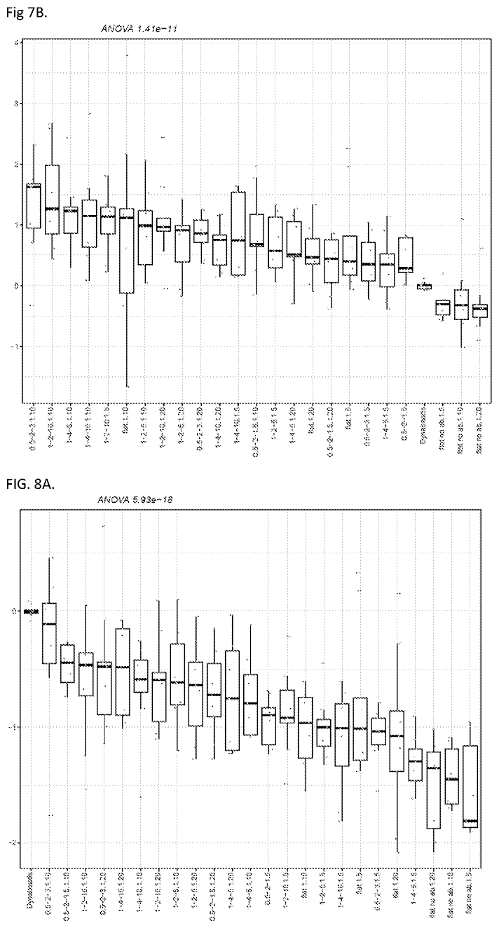

The results for the proliferation 3 days after the extraction and 7 days after the extraction are shown schematically in FIGS. 6A and 6B, respectively. In the figures, the z-score normalized box and whiskers plot is demonstrated, ranked by the highest to lowest in left-to-right order. Proliferation after 3 (FIG. 6(a)) and 7 (FIG. 6(b)) days is shown, being the result of dividing the total number of cells originating from the measured well—by 6×10⁵ (the number of cells aliquoted to this well on day 1). The number of cells originated from a specific well included calculation of the well split during the requisite number of days and cells that were taken for a specific assay. The feature is compared among all tested activation surfaces, across the x-axis. For each surface three replicates for three human donors were evaluated (n=9). The magnitude of for each measurement was Z-score normalized (y-axis), and the surfaces ranked from highest to lowest activation from left to right, based on the median measurement across replicates and donors. Specifically, the mean value measured for Dynabeads reference was subtracted and the relative measurements were scaled by dividing with the standard deviation of the measurement across all the tested surfaces. In the figures, the surfaces are labeled with codes, as elaborated above. The control groups are represented by the labels “Dynabeads” representing Dynabeads™, “flat no ab. 1.10” representing flat PDMS surface made of 1:10 ratio polymer and no antibodies attached, “flat no ab. 1.20” representing flat PDMS surface made of 1:20 ratio polymer and no antibodies attached, “flat no ab. 1.5” representing flat PDMS surface made of 1:5 ratio polymer and no antibodies attached, “flat 1.10” representing flat PDMS surface made of 1:10 ratio polymer with antibodies attached, “flat 1.20” representing flat PDMS surface made of 1:20 ratio polymer with antibodies attached, and “flat 1.5” representing flat PDMS surface made of 1:5 ratio polymer with antibodies attached. The p value of ANOVA was 0.008 at Day 3 and 3.89×10⁻¹¹ on Day 7, indicating statistically significant differences between the groups.

The activation of CD-3 positive cells was followed using the same markers for T cell activation, namely, CD69 and CD107a. The activation was analyzed on Day 1 with flow cytometry. DAPI negative and FITC-conjugated anti-human CD3 (UCHT1) was used to select live CD3⁺ cells, while APC-Cy7-conjugated anti-human CD69 (FN50) and perCP-Cy5.5-conjugated CD107a (H4A3) were used to stain CD69 and CD107a respectively.

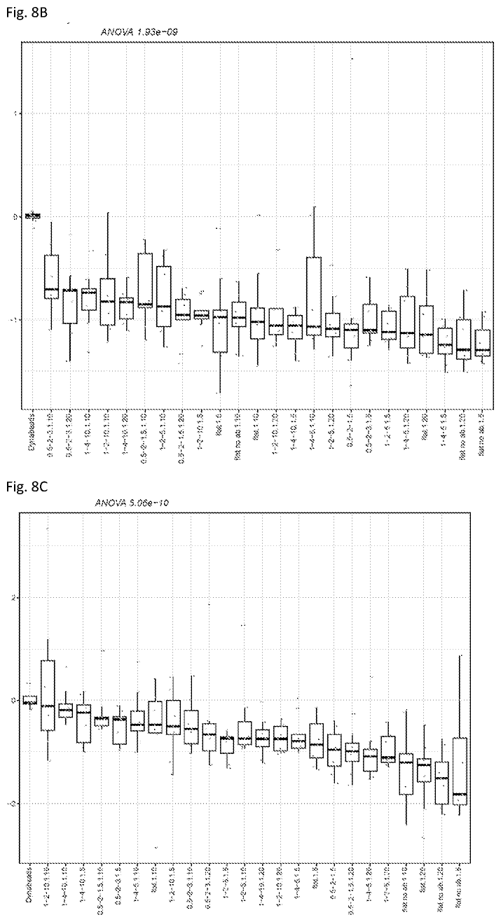

The results for day 1 for both markers, CD69 and CD107a, are presented in FIGS. 7A and 7B, respectively. The p value of ANOVA was 4.93×10⁻⁸ for CD69 and 1.41×10⁻¹¹ for CD107a, indicating statistically significant differences between the groups. The data has been processed as described for FIG. 6 above.

The exhaustion of the cells was determined on Day 3, and Day 7, by flow cytometry, detecting the exhaustion markers TIM3 (short-term exhaustion) and PD-1 (long-term exhaustion). DAPI negative and FITC-conjugated anti-human CD3 (UCHT1) was used to select live CD3+ cells, while APC-conjugated anti-human TIM3 (A18087E) and PE-conjugated anti-human PD-1 (EH12.2H7) were used to stain TIM3 and PD-1 respectively.

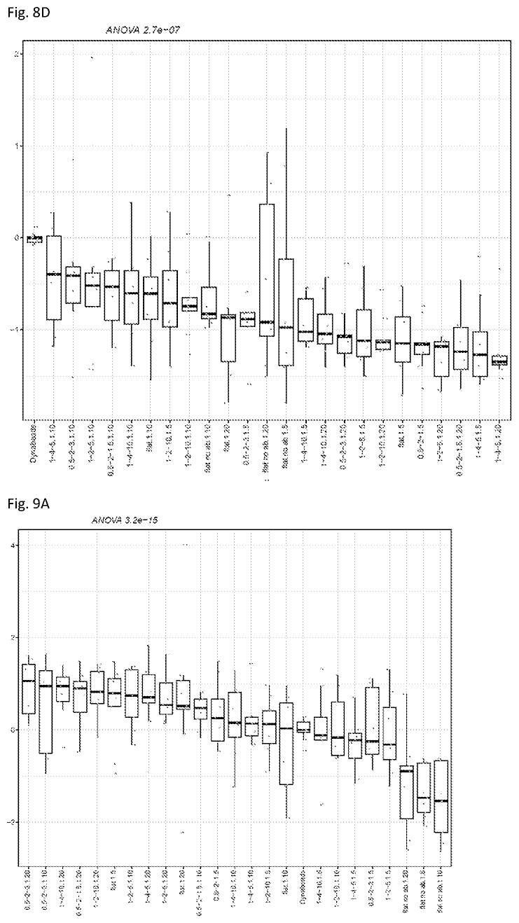

The results are presented in FIGS. 8A-8D, for TIM 3 on day 3, PD-1 on Day 3, TIM3 on Day 7, and PD-1 on Day 7, respectively. The results indicate the expression of these proteins on the T cell membrane and in accordance are directly associated with the exhaustion state of the T cells. The data has been processed as described for FIG. 6 above.

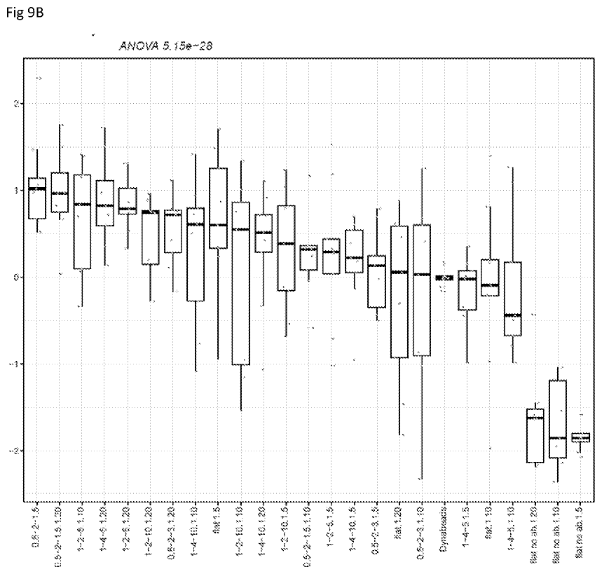

Transfection efficiency and stability was assessed on Day 3 and Day 7, by flow cytometry, detecting the fluorescent reporter gene product eGPF. The results are presented in FIGS. 9A and 9B, for the days 3 and 7, respectively. The data has been processed as described for FIG. 6 above. The results indicate that while T cells activated with Dynabeads™ demonstrate relatively high levels of exhaustion on day 3 and day 7 after activation, this exhaustion is substantially lower for T cells activated with PDMS brushes. The conclusion can therefore be made that the way T cells are initially activated affects their exhaustion in the long term.

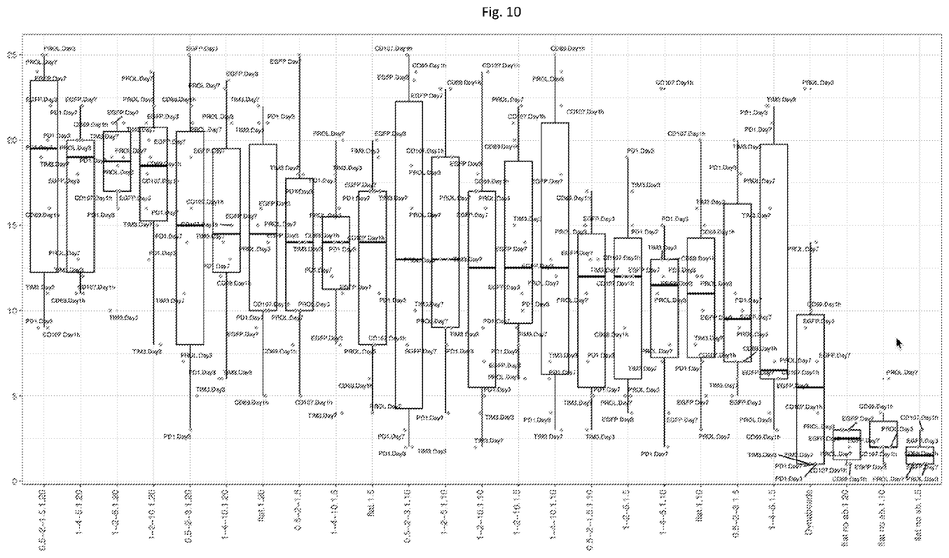

The pooled analysis of all the tested parameters was performed. The combined ranking of the surfaces was performed based on the z-scores of each individual test. With 25 compared activation surfaces for reach time-point specific activation feature, the surfaces were ranked from highest (rank-25) to lowest (rank=1). For each surface the relative ranking (1-25) for reach time-point-specific activation feature (y-axis) was visualized. The ranking across all features provides a global estimate of the surface performance, with surfaces ordered from highest to lowest activation from left to right, based on the median ranking across all activation features. The result is presented in FIG. 10.

The results clearly demonstrate that the micro-pillared surfaced perform superiorly to the Dynabeads™ golden standard, and to the flat unpatterned surfaces, and softer micro-pillars may be more preferable for more effective CAR T cell production. Specifically, it seems that the bulk modulus of the activating surface plays the key role in the activation of T cells. This can be illustrated by the fact that all the 1:20 surfaces (i.e. having the lowest tested bulk modulus) are in the in seven leading places in z-score analysis. Yet, an additional important factor in T cell activation is the surface topography. Specifically, within the seven “1:20” surfaces, the flat one got the lowest z score.

### Example 6

### Effect of the Activated PBMC on Patient-Derived Xenografts

To evaluate the effect of the cells activated as described in Example 5, patient-derived xenografts were incubated in presence of the cells, and the immunologic response in form of interferon-gamma secretion was measured.

Following Helsinki Committee approval, tumor biopsy samples LSE19 (human small cell lung carcinoma) were collected from the Soroka Medical Center and implanted subcutaneously in the dorsal flank of immune-deficient NOD scid gamma (NSG) mice. The animals were housed in standard microisolator cages with a 12-h light cycle and food and water. The tumor size was monitored periodically every 2-3 days using a digital caliper by measuring length and width of the tumor. Animals were monitored for weight loss every 3 days. After the xenografts reached the size of over 500 mm³, they were excised and sectioned into 2×2×2 mm tumor explants. The explants were incubated in 48 well flat bottom plate for 18-20 hours with either 20,000 or 50,000 PBMCs activated on either flat surfaces coated with anti-CD3 and anti-CD28, Dynabeads (commercially coated with anti CD3 and anti CD28), 1-2-5[1:20], or 0.5-2-1.5[1:20] PDMS micro-brushes surfaces coated with anti CD3 and anti CD28. The incubation was performed in NutriT™ media supplemented with 1 mM sodium pyruvate, 2 mM L-glutamine, 0.1 mM MEM nonessential amino acids, 1% penicillin/streptomycin, 10 mM HEPES (Life Technologies, Waltham, MA, USA), 30 IU of IL-2 (200-02-500UG, PeproTech, Cranbury, NJ, USA). After the incubation, the supernatant from the wells was collected and subjected to analysis using a standard IFN-γ ELISA assay (ELISA MAX, Biolegend, San Diego, CA, USA).

The tumor explants were subjected to Ki67 proliferation marker analysis by immunohistochemistry, as follows. The tissue samples were fixed in a 4% paraformaldehyde (PFA) solution for 24 hours at room temperature. After fixation, automated tissue processing machinery (Leica Biosystems, Nubloch, Germany) was employed to create FFPE blocks from these explants. Subsequently, using 3-mm T-Sue™ punch needle (Simport, Beloeil, QC, Canada), tissue microarray (TMA) blocks were prepared from the donor FFPE blocks. Each TMA block contained a maximum of 24 tissue explants. Paraffin-embedded tissue TMA blocks were then sectioned into 5 μm slices using a microtome. Before testing, the tissue sections were deparaffinized by immersing them in xylene. To block endogenous peroxidase activity, the sections were treated with a 3% hydrogen peroxide solution for 20 minutes, followed by rinsing in water for 5 minutes. Antigen retrieval was performed by immersing the sections in citrate buffer (pH 6) and heating them in a water bath at 95° C. for 30 minutes. After antigen retrieval, the sections were blocked for 1 hour at room temperature using a blocking solution consisting of phosphate-buffered saline (PBS) with 0.1% Tween and 5% bovine serum albumin (BSA). The sections were then incubated with primary antibodies overnight at 4° C. The Ki67 anti-human primary antibody (1:200 dilution) was diluted in the blocking solution according to the manufacturer's instructions. Following primary antibody incubation, the sections were processed using an ABC kit (VECTASTAIN Cat. VE-PK-6200) for color detection. The ABC kit utilizes a peroxidase-based enzymatic reaction to visualize the antigen-antibody complex. The sections were counterstained with hematoxylin to visualize cellular nuclei. Finally, the sections were mounted using a mounting medium (VectaMount™ permanent mounting medium) to preserve the staining. The stained slides were scanned using a Panoramic™ Scanner (3DHISTECH, Budapest, Hungary) to capture high-resolution digital images of the tissue sections. The scanned images were then analyzed using Panoramic™ viewer software to assess and quantify the immunohistochemical staining patterns. The results indicated a statistically significant decrease in cell proliferation in the micropatterned activating surfaces relative to the controls.

The gamma interferon release results indicated that with 50,000 cells the response was intense and no significant difference was observed between the groups. Conversely, with lower number of cells, statistically significant differences were observed. The results are presented in FIG. 10. It can be readily seen that the cells that underwent activation through 1-2-5[1:20] PDMS surface showed more robust response to the tumor compared to the cells that were activated using Dyna beads (DB) for all donors, as evidenced by the concentration of IFN-γ in the supernatant.

### Example 7

### Effect of the PBMC Activation on Transduction Efficiency by Electroporation

To evaluate the effect PBMC activation on transduction efficiency by electroporation, 1 million PBMCs were suspended in 100 μl of OptiMEM™ and were added into the cuvette of NEPA21 Super Electroporator. For Dynabeads™, magnets were used to remove the beads before performing electroporation. Efficiency and toxicity of electroporation were measured. The electroporation was performed either with protein (Cas9 tagged with eGFP protein (IDT company; 170 μg per 1×10⁶ cells)), or with DNA (2 μg of double-stranded eGFP DNA). Each was added to a different experimental cuvette. Electroporation was performed by Super Electroporator NEPA21 type 2 (NepaGene™) according to the manufacturer's instructions.

The electroporation settings were as follows: poring pulse: 175 V, pulse time 3.5 ms, pulse interval 50 ms, with 2 pulses and decay rate of 10; transfer pulse: 20 V, pulse time 50 ms, pulse interval 50 ms, with 5 pulses and decay rate of 40.

After electroporation, the cells were seeded to 1 ml of complete NutriT in 24 well plate. The electroporation efficiency was analyzed by flow cytometer after 2 h for Cas9 tagged eGFP and 24 h later for double stranded eGFP. The cells were stained with DAPI and PE anti-human-CD3 for the flow cytometry.

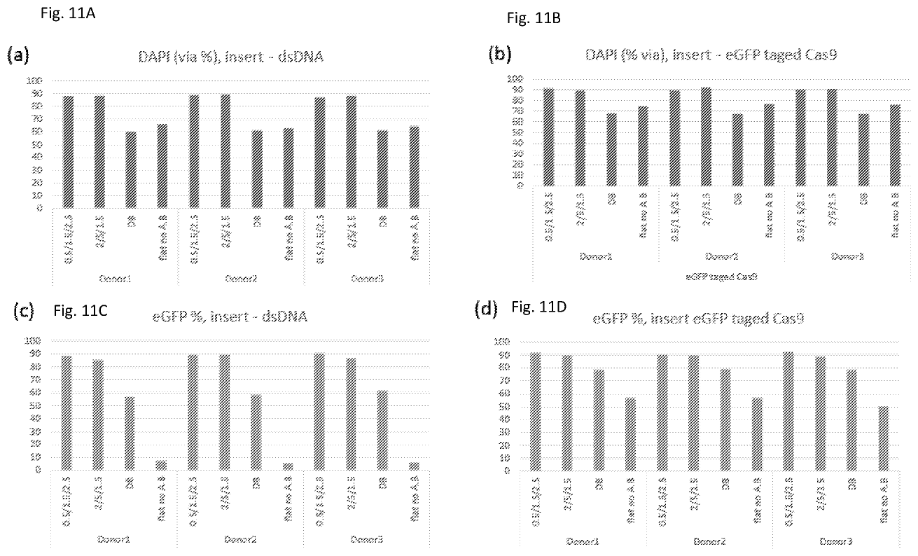

The results are presented in FIG. 11A-11D. The viability of the cells after the electroporation is demonstrated in FIGS. 11A and 11B, for dsDNA and Cas9-transfected cells, respectively, and FIGS. 11C and 11D demonstrate the transfection efficiency for the same cells. The results indicate that the flat surface-induced PBMC are less prone to transfection with dsDNA, whereas the tested activation surfaces outperformed both the Dynabeads and the flat surface controls both in the tolerability to electroporation and in the transfection efficiency.

### Example 8

### Effect of the Activated CAR T Cells on Cancer Cell Line

The T cells were activated as described in Example 5 on the following surfaces were used: 1-2-5[1:20], 1-4-5[1:20], 0.5-2-1.5[1:20], 1-2-10[1:20], 1-4-10[1:20], and 0.5-2-3 [1:20], as well as control surfaces of Dynabeads™, flat [1:20], and uncoated flat [1:20]. Following activation, CAR T cells were generated by transducing anti-HER2 CAR and cultured for 7 days.

Target HER2⁺ breast carcinoma cells resistant to trastuzumab JIMT1 were washed with PBS and labeled with membrane stain DiD (1,1′-dioctadecyl-3,3,3′,3′-tetramethylindodicarbocyanine, 4-chlorobenzenesulfonate salt, 1:250) for 15 minutes at 37° C. The cells were washed in PBS, resuspended in high glucose DMEM, and incubated for 20 minutes at 37° C. for recovery. Thereafter, 50,000 target cells/well were then seeded in 96 well plate. After about 1 hour, 100,000 pre-activated CAR-T cells were washed with PBS, suspended in NutriT™ (without IL2), and seeded onto the target cells. The plate was incubated for 3 hours at 37° C., and then centrifuged and the cells were detached by adding Versene™ solution (ThermoFisher, 0.48 mM (0.2 g/L) of tetrasodium EDTA in PBS)). The cells were washed with ×1 PAF, and the dead cells were stained with DAPI. The target JIMT1 cell viability was assessed by flow cytometry.

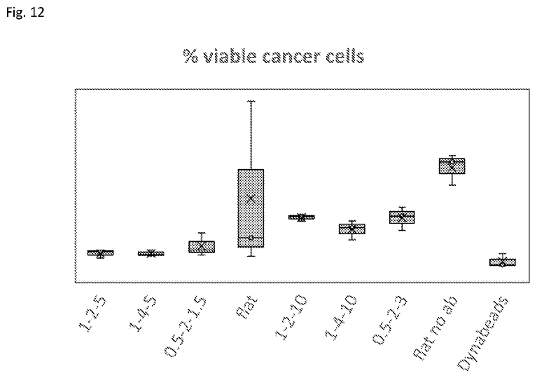

The results are presented in FIG. 12. It can be readily seen that apparently shorter micropatterned activation surfaces outperformed the flat controls and were at least on par with the Dynabeads™. However, one should consider that subset of T cells proliferating is higher in the micropatterned activation surfaces, as seen in the example 5 above, than in the Dynabeads™, the subset of fully differentiated T cells obtained from the micropatterned activated surfaces was sufficient to effect the same anticancer effect as in the whole collective obtained from the Dynabeads™.

1. A micropatterned surface comprising a base and a plurality of elongated elements disposed outwards essentially perpendicular to said base, said micropatterned surface consisting essentially of a soft elastomer polymer having a bulk elastic modulus of between 0.01 and 100 mega-Pascals, wherein said elongated elements are characterized by a bending stiffness of between 0.7 micro-Pascal*meter and 8 Pascals*meter, by a length of between 0.5 and 15 micrometers, and by a thickness of between 0.25 and 3 micrometers, and further wherein said elongated elements are placed in a density of between 25,000 and 4,000,000 per square millimeter, and optionally wherein said surface is sterile.

2. The micropatterned surface according to claim 1, characterized by: i) said soft elastomer having a bulk elastic modulus of between 0.1 and 5 mega-Pascals, ii) said elongated elements having bending stiffness of between 7 micro-Pascal*meter and 450 milli-Pascals*meter, iii) said elongated elements having the length of between 1.5 and 5 micrometers, or iv) a combination of any two or all three of the above.

3. (canceled)

4. (canceled)

5. The micropatterned surface according to claim 1, wherein said density is between 50,000 and 275,000 micro-pillars per square millimeter.

6. The micropatterned surface according to claim 1, wherein said soft elastomer is a poly(dimethyl siloxane), a poly(acrylamide), or a thermoplastic elastomer.

7. The micropatterned surface according to claim 1, coated with a linking moiety, optionally wherein said linking moiety is a bifunctional molecule comprising a first binding domain and a second binding domain, wherein said first binding domain bound to the surface of said polymer and said second binding domain being capable of binding a biologically active moiety, and further optionally wherein said linking moiety is 3-(aminopropyl)-triethoxysilane or thiol-polyethylene glycol-biotin.

8. (canceled)

9. (canceled)

10. The micropatterned surface according to claim 1, functionalized with a biologically active moiety, wherein said biologically active moiety is a ligand to a molecule expressed on the surface of a living cell, optionally wherein said living cell is a cytotoxic lymphocyte, and further optionally wherein said cytotoxic lymphocyte is a T cell selected from the group consisting of CD8⁺ T cells, CD4⁺ T cells, γδ-T cells, and natural killer T cells.

11. (canceled)

12. (canceled)

13. The micropatterned surface according to claim 10, wherein said biologically active moiety is characterized by binding to and activating and/or stimulating at least one of a T cell receptor (TCR)-CD3 complex and/or a CD28 molecule on the surface of said living cell, optionally wherein said biologically active moiety is an anti-CD3 antibody and/or an anti-CD28 antibody.

14. (canceled)

15. The micropatterned surface according to claim 1, further functionalized with an auxiliary moiety, optionally wherein said auxiliary moiety is an albumin.

16. (canceled)

17. The micropatterned surface according to claim 1, wherein said soft elastomer is poly(dimethyl siloxane) comprising between 2 and 25% of cross-linking moiety.

18.-27. (canceled)

28. A method for expanding cytotoxic lymphocytes using a micropatterned surface as defined in claim 1, said method comprising: (a) providing a cell collective comprising cytotoxic lymphocytes isolated from a biological sample obtained from a donor; (b) contacting said cell collective isolated in step (a) with a micropatterned surface as defined in claim 1, to effect the activation of said cytotoxic lymphocytes in said cell collective; (c) recovering said cell collective obtained in step (b) comprising activated cytotoxic lymphocytes from said micropatterned surface, and (d) culturing said collective, in the presence of a cytokine, thereby obtaining an expanded population of cytotoxic lymphocytes in said cells collective, and optionally further comprising isolating said cytotoxic lymphocytes from said cells collective, and further optionally wherein said contacting (b) is performed in presence of at least one cytokine, which may be same or different from said cytokine used in step (d), further comprising isolating said cytotoxic lymphocytes from said cells collective, and further optionally wherein said contacting (b) is performed in presence of at least one cytokine, which may be same or different from said cytokine used in step (d).

29. (canceled)

30. The method for expanding cytotoxic lymphocytes according to claim 28, wherein said at least one cytokine is selected from the group consisting of IL-1, IL-2, IL-4, IL-6, IL-7, IL-10, IL-12, IL-15, IL-18, and TNF.

31. (canceled)

32. The method for expanding cytotoxic lymphocytes according to claim 28, wherein said method is characterized by at least one of i) contacting is performed for a time period of between 15 minutes and 72 hours, optionally wherein said contacting is performed for a time period of between 2 and 8 hours, ii) wherein said culturing of said cytotoxic lymphocytes is carried out for a time period of between about 3 days to about 12 days, optionally wherein said time period is between about 4 days and about 7 days, or iii) both steps i) and ii) above.

33. (canceled)

34. (canceled)

35. (canceled)

36. The method for expanding cytotoxic lymphocytes according to claim 28, wherein said cytotoxic lymphocytes are CD8⁺ T cells, CD4⁺ T cells, γδ-T cells, or natural killer T cells, optionally wherein said micropatterned surface comprises a ligand that is characterized by binding to and activating and/or stimulating at least one of a T cell receptor (TCR)-CD3 complex and/or a CD28 molecule on the surface of said cytotoxic lymphocytes, and further optionally wherein said ligand is an anti-CD3 antibody and/or an anti-CD28 antibody.

37. (canceled)

38. (canceled)

39. The method for expanding cytotoxic lymphocytes according claim 28, further comprising genetically modifying said cytotoxic lymphocytes to express a chimeric antigen receptor.

40. The method for expanding cytotoxic lymphocytes according to claim 28, further comprising enriching said cytotoxic lymphocytes for an antigen-specific cell population, optionally wherein said enriching for T cells is performed after said isolation from said biological sample but prior to said contacting with said micropatterned surface.

41. (canceled)

42. The method for expanding cytotoxic lymphocytes according to claim 28, wherein said micropatterned surface is functionalized with a first ligand characterized by binding to and activating a TCR-CD3 complex and a second ligand characterized by binding to and activating a CD28 molecule on the surface of said cytotoxic lymphocyte, said contacting is performed for a time interval of between 20 and 28 hours, wherein said cytokine of step (d) is IL-2, and wherein said culturing is for a time interval of between 4 days and 7 days, optionally wherein said method further comprises genetically modifying said cells collective comprising cytotoxic lymphocytes isolated in step (a) to express a chimeric antigen receptor (CAR), and further optionally wherein said genetically modifying comprises any one of steps of electroporating said cells in presence of a Cas9 protein bearing CAR genetic material or in presence of double-stranded DNA encoding for CRISPR-Cas9 enzymatic system bearing said CAR genetic material, or transfecting said cells with a suitable viral carrier loaded with genetic material encoding said CRISPR-Cas9 and said CAR.

43. (canceled)

44. (canceled)

45. The method for expanding cytotoxic lymphocytes according to claim 28, wherein said cytotoxic lymphocytes are tumor infiltrating lymphocytes isolated from a tumor biopsy obtained from a cancer patient in need of treatment.

46. A method of treating or preventing a disease in a subject in need thereof comprising administering to said subject an effective amount cytotoxic lymphocytes obtained by using a micropatterned surface according to claim 1, wherein said cytotoxic lymphocytes are derived from said subject, and wherein said disease is a cancer, an inflammatory disease, or an infection.

47. The method of treating or preventing a disease according to claim 46, wherein said expanded population of cytotoxic lymphocytes comprise CAR T cells.

48. The method of treating or preventing a disease according to claim 46, wherein said expanded population of cytotoxic lymphocytes comprise tumor infiltrating lymphocytes.

49-53. (canceled)
